(19) United States
(12) Patent Application Publication
(10) Pub. No.: US 2022/0393238 A1
(43) Pub. Date: Dec. 8, 2022

(54) COMPOSITION

(71) Applicant: Mexichem Fluor S.A. de C.V., San Luis Potosi (MX)

(72) Inventors: Andrew Sharratt, Runcorn (GB); Ira Saxena, Runcorn (GB); Miodrag Oljaca, Concord, MA (US)

(21) Appl. No.: 17/765,736

(22) PCT Filed: Oct. 2, 2020
(86) PCT No.: PCT/EP2020/077746

§ 371 Date: Mar. 31, 2022

Related U.S. Application Data
(60) Provisional application No. 62/909,981, filed on Oct. 3, 2019.

(30) Foreign Application Priority Data
Nov. 7, 2019 (GB) 1916217.1
Jan. 22, 2020 (GB) 2000928.8
Feb. 28, 2020 (GB) 2002865.0

(51) Int. Cl. H01M 10/0567; C07C 43/17; C07C 41/08; H01M 10/0568; H01M 10/0569; H01M 10/42; H01M 10/0525

(52) U.S. Cl. CPC H01M 10/0567; C07C 43/17; C07C 41/08; H01M 10/0568; H01M 10/0569; H01M 10/4235; H01M 10/0525; H01M 2300/0034; H01M 2300/0037

(57) ABSTRACT

Use of a compound of Formula (I) in a nonaqueous battery electrolyte formulation (1) wherein R is a fluorinated alkyl group and X is selected from the group consisting of F, Cl, H, CF₃, and C₁ to C₆ alkyl which may be at least partially fluorinated and —OR group can be cis- or trans- to any other group X.

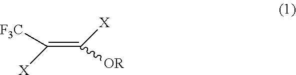

The present disclosure relates to nonaqueous electrolytic solutions for energy storage devices including batteries and capacitors, especially for secondary batteries and devices known as supercapacitiors.

There are two main types of batteries; primary and secondary. Primary batteries are also known as non-rechargeable batteries. Secondary batteries are also known as rechargeable batteries. A well-known type of rechargeable battery is the lithium-ion battery. Lithium-ion batteries have a high energy density, no memory effect and low self-discharge.

Lithium-ion batteries are commonly used for portable electronics and electric vehicles. In the batteries lithium ions move from the negative electrode to the positive electrode during discharge and back when charging.

Typically, the electrolytic solutions include a nonaqueous solvent and an electrolyte salt plus additives. The electrolyte is typically a mixture of organic carbonates such as ethylene carbonate, propylene carbonate, fluoroethylene carbonate and dialkyl carbonates containing a lithium ion electrolyte salt. Many lithium salts can be used as the electrolyte salt and common examples include lithium hexafluorophosphate (LiPF₆), lithium bis (fluorosulfonyl) imide “LiFSI” and lithium bis(trifluoromethanesulfonyl)imide (LiTFSI).

The electrolytic solution has to perform a number of separate roles within the battery.

The principal role of the electrolyte is to facilitate the flow of electrical charge between the cathode and anode. This occurs by transportation of metal ions within the battery from and or to one or both of the anode and cathode, where by chemical reduction or oxidation, electrical charge is liberated/adopted.

Thus the electrolyte needs to provide a medium which is capable of solvating and/or supporting the metal ions.

Due to the use of lithium electrolyte salts and the interchange of lithium ions with lithium metal; which is very reactive with water, as well as the sensitivity of other battery components to water; the electrolyte is usually non-aqueous.

Additionally the electrolyte has to have suitable rheological properties to permit/enhance the flow of ions therein; at the typical operating temperature to which a battery is exposed and expected to perform.

Moreover the electrolyte has to be as chemically inert as possible. This is particularly relevant, in the context of the expected lifetime of the battery, in regard to internal corrosion within the battery (e.g. of the electrodes and casing) and the issue of battery leakage. Also of importance within the consideration of chemical stability is flammability. Unfortunately typical electrolyte solvents can be a safety hazard since they often comprise a flammable material.

This can be problematic as in operation when discharging or being discharged, batteries may accumulate heat. This is especially true for high density batteries such as lithium ion batteries. It is therefore desirable that the electrolyte displays a low flammability, with other related properties such as a high flash point.

It is also desirable that the electrolyte does not present an environmental issue with regard to disposability after use or other environmental issue such as global warming potential.

It is an object of the present invention to provide a nonaqueous electrolytic solution, which provides improved properties over the nonaqueous electrolytic solution of the prior art.

### Use Aspects

According to a first aspect of the invention there is provided the use of a compound of Formula 1 in a nonaqueous battery electrolyte formulation. Preferably the composition comprising a compound of formula 1 is used in a lithium ion battery.

According to a second aspect of the invention there is provided the use of a nonaqueous battery electrolyte formulation comprising a compound of Formula 1 in a battery.

### Composition/Device Aspects

According to a third aspect of the invention there is provided a battery electrolyte formulation comprising a compound of Formula 1.

According to a fourth aspect of the invention there is provided a formulation comprising a metal ion and a compound of Formula 1, optionally in combination with a solvent.

According to a fifth aspect of the invention there is provided a battery comprising a battery electrolyte formulation comprising a compound of Formula 1.

### Method Aspects

According to a sixth aspect of the invention there is provided a method of reducing the flash point of a battery and/or a battery electrolyte formulation, comprising the addition of a formulation comprising a compound of Formula 1.

According to a seventh aspect of the invention there is provided a method of powering an article comprising the use of a battery comprising a battery electrolyte formulation comprising a compound of Formula 1.

According to an eighth aspect of the invention there is provided a method of retrofitting a battery electrolyte formulation comprising either (a) at least partial replacement of the battery electrolyte with a battery electrolyte formulation comprising a compound of Formula 1 and/or (b) supplementation of the battery electrolyte with a battery electrolyte formulation comprising a compound of Formula 1.

According to a ninth aspect of the invention there is provided a method of preparing a compound of method of preparing a compound of a compound of Formula 1 by reacting a compound of Formula 2a and/or Formula 2b

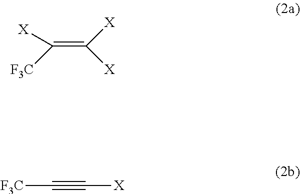

with an alcohol ROH; under basic reaction conditions at elevated temperature and pressure.

In formula 2a X is halogen or —CF₃ with the proviso that at least one X is H. Most preferably at least one X is halogen and at least one X is H and wherein these where at least one X is halogen and one X is hydrogen these groups are trans to each other.

In formula 2b X is hydrogen or —CF₃.

According to a tenth aspect of the invention there is provided a method of preparing a battery electrolyte formulation comprising mixing comprising a compound of Formula 1 with a lithium containing compound.

According to an eleventh aspect of the invention there is provided a method of improving battery capacity/charge transfer within a battery/battery life/etc by the use of a compound of Formula 1.

### Compound of Formula 1

In reference to all aspects of the invention the preferred embodiment of Formula (1) is below

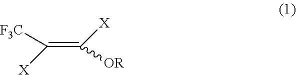

wherein
R is a fluorinated alkyl group and the stereochemistry of the —OR group can be either cis- or trans- to any other function and X is selected from the group consisting of F, Cl, H, CF₃, and C₁ to C₆ alkyl which may be at least partially fluorinated.

Alternatively, and also in reference to all aspects of the invention an alternate embodiment of Formula (1) is below

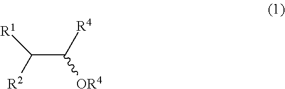

wherein
R¹ is selected from the group consisting of F, Cl, H, CF₃, and C₁ to C₆ alkyl which may be at least partially fluorinated;
R² is selected from the group consisting of F, Cl, H, CF₃, and C₁ to C₆ alkyl which may be at least partially fluorinated;

R³ is selected from the group consisting of F, Cl, H, CF₃, and C₁ to C₆ alkyl which may be at least partially fluorinated;

R⁴ is selected from the group consisting of and C₁ to C₁₂ alkyl which may be at least partially fluorinated;

wherein at least one of R¹ to R⁴ is or comprises F and the stereochemistry of the —OR⁴ group can be cis- or trans- to any other function.

It is to be noted that the ninth aspect of the invention shall be taken to apply to both embodiments of the Formula (1).

### Advantages

In the aspects of the invention the electrolyte formulation has been found to be surprisingly advantageous.

The advantages of using compounds of Formula 1 in electrolyte solvent compositions manifest themselves in a number of ways. Their presence can reduce the flammability of the electrolyte composition (such as when for example measured by flashpoint). Their oxidative stability makes them useful for batteries required to work in harsh conditions and they are compatible with common electrode chemistries and can even enhance the performance of these electrodes through their interactions with them.

Additionally, electrolyte compositions comprising compounds of formula 1 have been found to have superior physical properties including low viscosity and a low melting point, yet a high boiling point with the associated advantage of little or no gas generation in use. The electrolyte formulation has been found to wet and spread extremely well over surfaces particularly fluorine containing surfaces; this is postulated to result from a beneficial a relationship between its adhesive and cohesive forces, to yield a low contact angle.

Furthermore, electrolyte compositions that comprise compounds of Formula 1 have been found to have superior electro-chemical properties including improved capacity retention, improved cyclability and capacity, improved compatibility with other battery components e.g. separators and current collectors and with all types of cathode and anode chemistries including systems that operate across a range of voltages and especially high voltages and which include additives such as silicon. In addition, the electrolyte formulations display good solvation of metal (e.g. lithium) salts and interaction with any electrolyte solvents present.

Preferred features relating to the aspects of the invention follows below.

### Preferred Compounds

Preferred examples of compounds of the first embodiment of Formula 1

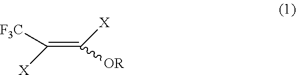

are where:—
R is CH₂CF₃, CH₂CF₂CF₂CHF₂ or CH(CF₃)₂; and

### X is H.

Preferred features of the alternate embodiment of the compound of Formula 1

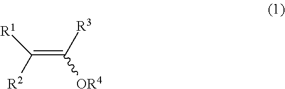

are as in the following numbered paragraphs.

Paragraph 1—the compound of Formula 1, wherein preferably at least two or three of R¹ to R⁴ is or comprises F; for example one or two of R¹ to R³ is or comprises F while R⁴ comprises F.

Paragraph 2—the compound of paragraph 1, wherein preferably only one of R¹ to R⁴ comprises C₁ to C₆ alkyl, whether unfluorinated or at least partially fluorinated.

Paragraph 3—the compound of paragraph 1 or 2, wherein preferably R² is selected from the group consisting of H, CF₃, and C₁ to C₆ alkyl which may be at least partially fluorinated.

Paragraph 4—the compound of paragraphs 1 to 3, wherein preferably R² is selected from the group consisting of H and CF₃.

Paragraph 5—the compound of paragraphs 1 to 4, wherein preferably R² is CF₃.

Paragraph 6—the compound of paragraphs 1 to 5, wherein preferably R⁴ is C₁ to C₆ alkyl which may be at least partially fluorinated;

Paragraph 7—the compound of paragraphs 1 to 6, wherein preferably R⁴ is C₁ to C₄ alkyl which may be at least partially fluorinated;

Paragraph 8—the compound of paragraphs 1 to 7, wherein preferably R⁴ is selected from the group consisting of ethyl, n-propyl, iso-propyl, n-butyl, iso-butyl and at least partially fluorinated derivatives thereof.

Paragraph 9—the compound of paragraphs 1 to 8, wherein preferably R⁴ is selected from the group consisting of CH₂CF₃, CH₂CH₂CF₃, CH₂CHFCF₃, CH₂CF₂CF₂CHF₂ and CH(CF₃)₂.

Paragraph 10—the compound of paragraphs 1 to 9, wherein preferably R¹ and R³ are independently selected from the group consisting of H, CF₃, and C₁ to C₆ alkyl which may be at least partially fluorinated.

Paragraph 11—the compound of paragraphs 1 to 10, wherein preferably R¹ and R³ are independently selected from the group consisting of H, CF₃, CH₂CF₃, CH₂CH₂CF₃, CH₂CHFCF₃, CH₂CF₂CF₂CHF₂ and CH(CF₃)₂.

Paragraph 12—the compound of paragraphs 1 to 11, wherein preferably R¹ and R³ are independently selected from the group consisting of H and CF₃.

Paragraph 13—the compound of paragraphs 1 to 12, wherein preferably R¹ and R³ is H.

Preferred examples of compounds of the alternate embodiment of the compound of Formula 1 are where:—

### R¹ is H,

R² is CF₃,

### R³ is H, and

R⁴ is CH₂CF₃, CH₂CF₂CF₂CHF₂ or CH(CF₃)₂.

### Electrolyte Formulation

Preferably the electrolyte formulation comprises 0.1 wt % to 99.9 wt % of a compound of Formula 1. Optionally the compound of Formula 1 is present (in the electrolyte formulation) in an amount of more than 1 wt %, optionally more than 5 wt %, optionally more than 10 wt %, optionally more than 15 wt %, optionally more than 20 wt % and optionally more than 25 wt %. Optionally the compound of Formula 1 is present (in the electrolyte formulation) in an amount of less than 1 wt %, optionally less than 5 wt %, optionally less than 10 wt %, optionally less than 15 wt %, optionally less than 20 wt % and optionally less than 25 wt %.

### Metal Salts

The nonaqueous electrolytic solution further comprises a metal electrolyte salt, typically present in an amount of 0.1 to 20 wt % relative to the total mass of the nonaqueous electrolyte formulation.

The metal salt generally comprises a salt of lithium, sodium, magnesium, calcium, lead, zinc or nickel.

Preferably the metal salt comprises a salt of lithium, such as those selected from the group comprising lithium hexafluorophosphate (LiPF₆), lithium hexafluoroarsenate monohydrate (LiAsF₆), lithium perchlorate (LiClO₄), lithium tetrafluoroborate (LiBF₄), lithium triflate (LiSO₃CF₃), lithium bis(fluorosulfonyl)imide (Li(FSO₂)₂N) and lithium bis(trifluoromethanesulfonyl)imide (Li(CF₃SO₂)₂N).

Most preferably the metal salt comprises LiPF₆. Thus in a most preferred variant of the fourth aspect of the invention there is provided a formulation comprising LiPF₆ and a compound of Formula 1, optionally in combination with a solvent

### Solvents

The nonaqueous electrolytic solution may comprise a solvent. Preferred examples of solvents include fluoroethylene carbonate (FEC) and/or propylene carbonate (PC), dimethyl carbonate (DMC), ethylmethyl carbonate (EMC) or ethylene carbonate (EC).

Where present the solvent makes up from 0.1 wt % to 99.9 wt % of the liquid component of the electrolyte.

### Additives

The nonaqueous electrolytic solution may include an additive.

Suitable additives may serve as surface film-forming agents, which form an ion permeable film on the surface of the positive electrode or the negative electrode. This can pre-empt a decomposition reaction of the nonaqueous electrolytic solution and the electrolyte salt occurring on the surface of the electrodes, thereby preventing the decomposition reaction of the nonaqueous electrolytic solution on the surface of the electrodes.

Examples of film-forming agent additives include vinylene carbonate (VC), ethylene sulfite (ES), lithium bis(oxalato)borate (LiBOB), cyclohexylbenzene (CHB) and ortho-terphenyl (OTP). The additives may be used singly, or two or more may be used in combination.

When present the additive is present in an amount of 0.1 to 3 wt % relative to the total mass of the nonaqueous electrolyte formulation.

### Battery

### Primary/Secondary Battery

The battery may comprise a primary (non-rechargeable) or a secondary battery (rechargeable). Most preferably the battery comprises a secondary battery.

A battery comprising the nonaqueous electrolytic solutions will generally comprise several elements. Elements making up the preferred nonaqueous electrolyte secondary battery cell are described below. It is appreciated that other battery elements may be present (such as a temperature sensor); the list of battery components below is not intended to be exhaustive.

### Electrodes

The battery generally comprises a positive and negative electrode. Usually the electrodes are porous and permit metal ion (lithium ions) to move in and out of their structures with a process called insertion (intercalation) or extraction (deintercalation).

For rechargeable batteries (secondary batteries), the term cathode designates the electrode where reduction is taking place during the discharge cycle. For lithium-ion cells the positive electrode (“cathode”) is the lithium-based one.

### Positive Electrode (Cathode)

The positive electrode is generally composed of a positive electrode current collector such as a metal foil, optionally with a positive electrode active material layer disposed on the positive electrode current collector.

The positive electrode current collector may be a foil of a metal that is stable at a range of potentials applied to the positive electrode, or a film having a skin layer of a metal that is stable at a range of potentials applied to the positive electrode. Aluminium (Al) is desirable as the metal that is stable at a range of potentials applied to the positive electrode.

The positive electrode active material layer generally includes a positive electrode active material and other components such as a conductive agent and a binder. This is generally obtained by mixing the components in a solvent, applying the mixture onto the positive electrode current collector, followed by drying and rolling.

The positive electrode active material may be a lithium (Li) containing transition metal oxide. The transition metal element may be at least one selected from the group consisting of scandium (Sc), manganese (Mn), iron (Fe), cobalt (Co), nickel (Ni), copper (Cu) and yttrium (Y). Of these transition metal elements, manganese, cobalt and nickel are the most preferred.

Some of the transition metal atoms in the transition metal oxide may be replaced by atoms of a non-transition metal element. The non-transition element may be selected from the group consisting of magnesium (Mg), aluminium (Al), lead (Pb), antimony (Sb) and boron (B). Of these non-transition metal elements, magnesium and aluminium are the most preferred.

Preferred examples of positive electrode active materials include lithium-containing transition metal oxides such as LiCoO₂, LiNiO₂, LiMn₂O₄, LiMnO₂, LiNi_{1-y}Co_{y}O₂ (0<y<1), LiNi_{1-y-z}Co_{y}Mn_{z}O₂ (0<y+z<1) and LiNi_{1-y-z}Co_{y}Al_{z}O₂ (0<y+z<1). LiNi_{1-y-z}Co_{y}Mn_{z}O₂ (0<y+z<0.5) and LiNi_{1-y-z}Co_{y}Al_{z}O₂ (0<y+z<0.5) containing nickel in a proportion of not less than 50 mol % relative to all the transition metals are desirable from the perspective of cost and specific capacity. These positive electrode active materials contain a large amount of alkali components and thus accelerate the decomposition of nonaqueous electrolytic solutions to cause a decrease in durability. However, the nonaqueous electrolytic solution of the present disclosure is resistant to decomposition even when used in combination with these positive electrode active materials.

The positive electrode active material may be a lithium (Li) containing transition metal fluoride. The transition metal element may be at least one selected from the group consisting of scandium (Sc), manganese (Mn), iron (Fe), cobalt (Co), nickel (Ni), copper (Cu) and yttrium (Y). Of these transition metal elements, manganese, cobalt and nickel are the most preferred.

Some of the transition metal atoms in the transition metal fluoride may be replaced by atoms of a non-transition metal element. The non-transition element may be selected from the group consisting of magnesium (Mg), aluminium (Al), lead (Pb), antimony (Sb) and boron (B). Of these non-transition metal elements, magnesium and aluminium are the most preferred.

A conductive agent may be used to increase the electron conductivity of the positive electrode active material layer. Preferred examples of the conductive agents include conductive carbon materials, metal powders and organic materials. Specific examples include carbon materials as acetylene black, ketjen black and graphite, metal powders as aluminium powder, and organic materials as phenylene derivatives.

A binder may be used to ensure good contact between the positive electrode active material and the conductive agent, to increase the adhesion of the components such as the positive electrode active material with respect to the surface of the positive electrode current collector. Preferred examples of the binders include fluoropolymers and rubber polymers, such as polytetrafluoroethylene (PTFE), polyvinylidene fluoride (PVdF) ethylene-propylene-isoprene copolymer and ethylene-propylene-butadiene copolymer. The binder may be used in combination with a thickener such as carboxymethylcellulose (CMC) or polyethylene oxide (PEO).

### Negative Electrode (Anode)

The negative electrode is generally composed of a negative electrode current collector such as a metal foil, optionally with a negative electrode active material layer disposed on the negative electrode current collector.

The negative electrode current collector may be a foil of a metal. Copper (lithium free) is suitable as the metal. Copper is easily processed at low cost and has good electron conductivity.

Generally, the negative electrode comprises carbon, such as graphite or graphene.

Silicon based materials can also be used for the negative electrode. A preferred form of silicon is in the form of nano-wires, which are preferably present on a support material. The support material may comprise a metal (such as steel) or a non-metal such as carbon.

The negative electrode may include an active material layer. Where present the active material layer includes a negative electrode active material and other components such as a binder. This is generally obtained by mixing the components in a solvent, applying the mixture onto the positive electrode current collector, followed by drying and rolling.

Negative electrode active materials are not particularly limited, provided the materials can store and release lithium ions. Examples of suitable negative electrode active materials include carbon materials, metals, alloys, metal oxides, metal nitrides, and lithium-intercalated carbon and silicon. Examples of carbon materials include natural/artificial graphite, and pitch-based carbon fibres. Preferred examples of metals include lithium (Li), silicon (Si), tin (Sn), germanium (Ge), indium (In), gallium (Ga), titanium, lithium alloys, silicon alloys and tin alloys. Examples of lithium based materials include lithium titanate (Li₂TiO₃)

As with the positive electrode, the binder may be a fluoropolymer or a rubber polymer and is desirably a rubbery polymer, such as styrene-butadiene copolymer (SBR). The binder may be used in combination with a thickener.

### Separator

A separator is preferably present between the positive electrode and the negative electrode. The separator has insulating properties. The separator may comprise a porous film having ion permeability. Examples of porous films include microporous thin films, woven fabrics and nonwoven fabrics. Suitable materials for the separators are polyolefins, such as polyethylene and polypropylene.

### Case

The battery components are preferably disposed within a protective case.

The case may comprise any suitable material which is resilient to provide support to the battery and an electrical contact to the device being powered.

In one embodiment the case comprises a metal material, preferably in sheet form, moulded into a battery shape. The metal material preferably comprises a number of portions adaptable be fitted together (e.g. by push-fitting) in the assembly of the battery. Preferably the case comprises an iron/steel-based material.

In another embodiment the case comprises a plastics material, moulded into a battery shape. The plastics material preferably comprises a number of portions adaptable be joined together (e.g. by push-fitting/adhesion) in the assembly of the battery. Preferably the case comprises a polymer such as polystyrene, polyethylene, polyvinyl chloride, polyvinylidene chloride, or polymonochlorofluoroethylene. The case may also comprise other additives for the plastics material, such as fillers or plasticisers. In this embodiment wherein the case for the battery predominantly comprises a plastics material a portion of the casing may additionally comprise a conductive/metallic material to establish electrical contact with the device being powered by the battery.

### Arrangement

The positive electrode and negative electrode may be wound or stacked together through a separator. Together with the nonaqueous electrolytic solution they are accommodated in the exterior case. The positive and negative electrodes are electrically connected to the exterior case in separate portions thereof

### Module/Pack

A number/plurality of battery cells may be made up into a battery module. In a battery module the battery cells may be organised in series and/or parallel. Typically these are encased in a mechanical structure.

A battery pack may be assembled by connecting multiple modules together in series or parallel. Typically battery packs include further features such as sensors and controllers including battery management systems and thermal management systems. The battery pack generally includes an encasing housing structure to make up the final battery pack product.

### End Uses

The battery of the invention, in the form an individual battery/cell, module and/or pack (and the electrolyte formulations therefor) are intended to be used in one or more of a variety of end products.

Preferred examples of end products include portable electronic devices, such as GPS navigation devices, cameras, laptops, tablets and mobile phones. Other preferred examples of end products include vehicular devices (as provision of power for the propulsion system and/or for any electrical system or devices present therein) such as electrical bicycles and motorbikes as well as automotive applications (including hybrid and purely electric vehicles).

The invention will now be illustrated with reference to the following non-limiting examples.

### EXAMPLES

### Example 1—Ether Preparation

The following steps were followed.
Potassium hydroxide (17.0 g, 250 mmol) was added slowly and with stirring to a solution of water (4.0 g) and alcohol (40 g) in a 100 ml Hastelloy C pressure vessel which was equipped with an inlet port for gases or liquids, pressure and temperature indicators and a cruciform gas entraining stirrer assembly.
The vessel was sealed, purged with nitrogen and pressure tested. The contents were then heated to 20-60° C. (depending on the reaction) with stirring at 1000 rpm
Once equilibrated, the unsaturated organofluorine feed component was added and the reaction conditions maintained until no further change was indicated by the pressure or temperature indicators.
The contents were recovered, washed with water and the organic fraction recovered.
The organic fraction was dried over anhydrous sodium sulphate and subjected to a simple “top and tail” distillation to remove lights and heavies.
The reaction products were then analysed by GC-MS.

| Expt. | E1 | E2 | E3 |
|---|---|---|---|
| Organofluorine feed | TFMA | TFMA | TFMA |
| water (g) | 4 | 4 | 4 |
| Alcohol (40 g) | CHF₂CCF₂CH₂OH | CF₃CH₂OH | (CF₃)₂CHOH |
| Product | Z—CF₃CH═CHOCH₂CF₂CHF₂ | Z—CF₃CH═CHOCH₂CF₃ | Z—CF₃CH═CHOCH(CF₃)₂ |
| Yield and Comments | 99.7%, reaction fast but | 90.3%, reaction very | KOH solubility in alcohol |
| | phase separation difficult | fast, easy separation, | very poor: care needed to |
| | with an emulsion phase | possibly some E- | avoid dehydration/ |
| | and signs of oligomers, | isomer, very clean | charring. KOH only to |
| | possibly some E-isomer | product with only | 11.7 g and water 9 g. |
| | along with some minor | minor impurities | Reaction very slow, (72 |
| | impurities | | hours). Difficult phase |
| | | | separation, significant |
| | | | emulsion phase with signs |
| | | | of oligomers. Crude |
| | | | product contained only |
| | | | traces of expected |
| | | | hexafluoroisopropyl ether |
| Mass Spectrum | m/z: 51(CHF₂), 69(CF₃), 75, 77, | m/z: 69(CF₃), 75, 77, | m/z: 69(CF₃), 75, 91, |
| | 91, 93, 95(CF₃CH═CH), | 83(CF₃CH₂), 91, 93, | 95(CF₃CH═CH), 99, 113, |
| | 125(CF₃CH═CHOCH₂), 207(M- | 95(CF₃CH═CH), 125((M- | 145, 193(M-69), 243(M-F), |
| | 19), 226(M) | 69, CF₃CH═CHOCH₂), | 262(M) |
| | | 175(M-19), 194(M) | |

| Expt. | E4 | E5 |
|---|---|---|
| Organofluorine feed | TFMA | TFMA |
| water (g) | 4 | 4 |
| Alcohol (40 g) | (CF₃)₂CHOH | CF₃CH₂CH₂OH |
| Product | Z—CF₃CH═CHOCH(CF₃)₂ | Z—CF₃CH═CHO(CH₂)₂CF₃ |
| Yield and Comments | Procedure adjusted: water | 77%, phase |
| | 2.5 g, HFIP 25 g, KOH 2.6 | separation very slow, |
| | g, Aliquat 336 0.1 g. | emulsion phase with |
| | Reaction still slow and left | signs of oligomers |
| | overnight. Phase | |
| | separation impossible so | |
| | volatiles recovered by | |
| | vacuum distillation. 9 g of a | |
| | free running clear organic | |
| | liquid recovered that | |
| | contained approximately 30 | |
| | area % of the desired | |
| | product | |
| Mass Spectrum | m/z: 69(CF₃), 75, 91, | m/z: 51(CHF₂), 69(CF₃) [symbol] |
| | 95(CF₃CH═CH), 99, 113, | 77, 91, 92, 93, 112, 18 [symbol] |
| | 145, 193(M-69), 243(M-F), | 19), 208(M) |
| | 262(M) | |

TFMA is trifluoromethyl acetylene [symbol]
indicates data missing or illegible when filed

### Example 2—Large Scale Ether Preparation

The basic procedure outlined in Example 1 was followed with larger batches (300-500 g) of the organofluorine feed component and the crude products were analysed by NMR spectroscopy.

| Expt. | E6 | E7 |
|---|---|---|
| Organofluorine | 1-Chloro-3,3,3- | 1-Chloro-3,3,3- |
| feed | trifluoropropene | trifluoropropene |
| component (g) | | |
| Product | 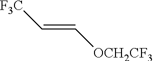 | 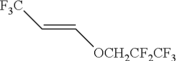 |
| Purity | ≥99.5 | ≥99.5 |
| ¹⁹F NMR(56 | δ −59.78 (CF₃CH═, dd, | δ −59.88 (CF₃CH═, d, |
| MHz) (CDCl₃ vs | J = 8.1, 2.1 Hz), −77.15 | J = 7.9), −127.55 (CF₂, tq, |
| PFB) | (CF₃CH₂, t, J = 8.1 Hz) | J = 11.9, 3.8 Hz), −141.13 |
| | | (CHF₂, (dt, J = 53.1, 3.6 |
| | | Hz.) |

### Example 3—Electrochemical Compatibility Testing

Electrochemical compatibility was assessed by cyclic voltammetry (CV) using a Gamry Instruments Potentiometer and a standard three electrode test cell. The working and counter electrodes were made of glassy carbon (area 0.071 cm²) with a platinum wire reference electrode. The basic electrolyte solution was 0.25 M tetrabutyl ammonium fluoroborate (TBAF) in acetonitrile (ACN) and the cell was referenced to ferrocene/ferrocenium (Fc/Fc⁺) couple at 0 V.

FIG. 1 shows three CV traces which serve to demonstrate the electrochemical compatibility of trifluoropropenyl ethers such as Product E6:
CV 1 0.25 M TBAF in ACN
CV 2 0.25 M TBAF in ACN+Product E6
CV 3 0.25 M TBAF in ACN+Propylene carbonate

### Example 4—Preparation of Electrolyte Compositions

1 M solutions of lithium hexafluorophosphate (LiPF₆)/lithium bis(fluorosulfonyl) ( ) in solvents comprising the products of E6 or E7 and various common electrolyte solvents were prepared and analysed by ¹⁹F NMR spectroscopy.

The compositions are shown in Tables 1 to 4 below. Tables 1 to 4 also contain a reference to the ¹⁹F NMR spectrum (see also page 21).

In Tables 1 to 4 the following abbreviations are used.
PC=propylene carbonate
FEC=fluoroethylene carbonate
EC=ethylene carbonate
EMC=ethyl methyl carbonate

All percentages are by weight.

TABLE 1

| | | % Additive | |
|---|---|---|---|
| Composition | Base | (CF₃CH═CHOCH₂CF₃) | FIG. |
| 1 | 95% 1M LiPF₆ in PC | 5% | 12a |
| 2 | 85% 1M LiPF₆ in PC | 15% | 12b |
| 3 | 75% 1M LiPF₆ in PC | 25% | 12c |
| 4 | 25% 1M LiPF₆ in PC | 75% | 12d |
| 5 | 95% 1M LiPF₆ in PC (90%) and | 5% | 13a |
| | FEC (10%) | | |
| 6 | 85% 1M LiPF₆ in PC (90%) and | 15% | 13b |
| | FEC (10%) | | |
| 7 | 25% 1M LiPF₆ in PC (90%) and | 75% | 13c |
| | FEC (10%) | | |
| 8 | 95% 1M LiPF₆ in EC (30%) and | 5% | 14a |
| | EMC (70%) | | |
| 9 | 85% 1M LiPF₆ in EC (30%) and | 15% | 14b |
| | EMC (70%) | | |
| 10 | 25% 1M LiPF₆ in EC (30%) and | 75% | 14c |
| | EMC (70%) | | |

TABLE 2

| | | % Additive | |
|---|---|---|---|
| Composition | Base | (CF₃CH═CHOCH₂CF₃) | FIG. |
| 11 | 95% 1M LiFSI in PC | 5% | 15a |
| 12 | 85% 1M LiFSI in PC | 15% | 15b |
| 13 | 25% 1M LiFSI in PC | 75% | 15c |
| 14 | 95% 1M LiFSI in PC (90%) and | 5% | 16a |
| | FEC (10%) | | |
| 15 | 85% 1M LiFSI in PC (90%) and | 15% | 16b |
| | FEC (10%) | | |
| 16 | 25% 1M LiFSI in PC (90%) and | 75% | 16c |
| | FEC (10%) | | |
| 17 | 85% 1M LiFSI in EC (30%) and | 15% | 17a |
| | EMC (70%) | | |
| 18 | 25% 1M LiFSI in EC (30%) and | 75% | 17b |
| | EMC (70%) | | |

TABLE 3

| | | % Additive | |
|---|---|---|---|
| Composition | Base | (CF₃CH═CHOCH₂CF₂CHF₂) | FIG. |
| 19 | 95% 1M LiPF₆ in PC | 5% | 18a |
| 20 | 85% 1M LiPF₆ in PC | 15% | 18b |
| 21 | 25% 1M LiPF₆ in PC | 75% | 18c |
| 22 | 95% 1M LiPF₆ in PC (90%) and | 5% | 19a |
| | FEC (10%) | | |
| 23 | 85% 1M LiPF₆ in PC (90%) and | 15% | 19b |
| | FEC (10%) | | |
| 24 | 25% 1M LiPF₆ in PC (90%) and | 75% | 19c |
| | FEC (10%) | | |
| 25 | 95% 1M LiPF₆ in EC (30%) and | 5% | 20a |
| | EMC (70%) | | |
| 26 | 85% 1M LiPF₆ in EC (30%) and | 15% | 20b |
| | EMC (70%) | | |
| 27 | 25% 1M LiPF₆ in EC (30%) and | 75% | 20c |
| | EMC (70%) | | |

TABLE 4

| | | % Additive | |
|---|---|---|---|
| Composition | Base | (CF₃CH═CHOCH₂CF₂CHF₂) | FIG. |
| 28 | 95% 1M LiFSI in PC | 5% | 21a |
| 29 | 85% 1M LiFSI in PC | 15% | 21b |
| 30 | 25% 1M LiFSI in PC | 75% | 21c |
| 31 | 95% 1M LiFSI in PC (90%) and | 5% | 22a |
| | FEC (10%) | | |
| 32 | 85% 1M LiFSI in PC (90%) and | 15% | 22b |
| | FEC (10%) | | |
| 33 | 25% 1M LiFSI in PC (90%) and | 75% | 22c |
| | FEC (10%) | | |
| 34 | 95% 1M LiFSI in EC (30%) and | 5% | 23a |
| | EMC(70%) | | |
| 35 | 85% 1M LiFSI in EC (30%) and | 15% | 23b |
| | EMC(70%) | | |
| 36 | 25% 1M LiFSI in EC (30%) and | 75% | 23c |
| | EMC(70%) | | |

### Flammability and Safety Testing

### Flash Point

Flashpoints were determined using a Miniflash FLP/H device from Grabner Instruments following the ASTM D6450 standard method:

| | Concentration (% wt) in standard electrolyte 1M LiPF₆ in | | | | | |
|---|---|---|---|---|---|---|
| | (30% Ethylenecarbonate & 70% ethyl methyl carbonate) | | | | | |
| | 0 | 2 | 5 | 10 | 30 | 100 |
| Component | Flashpoint (° C.) | | | | | |
| CF₃CH═CHOCH₂CF₃ | 32 ± 2 | 36 ± 2 | 35 ± 2 | 36 ± 1 | 43 ± 1 | not |
| (MEXI-1) | | | | | | detectable |
| CF₃CH═CHOCH₂CF₂CF₂H | 32 ± 2 | 35 ± 2 | 37 ± 1 | 38 ± 1 | 47 ± 2 | 61 ± 2 |
| (MEXI-2) | | | | | | |

These measurements demonstrate that the addition of the additives designated MEXI-1 and MEXI-2 significantly raised the flashpoint of the standard electrolyte solution.

### Self-Extinguishing Time

Self-extinguishing time was measured with a custom-built device that contained an automatically controlled stopwatch connected to an ultraviolet light detector:
The electrolyte to be examined (500 μL) was applied to a Whatman GF/D (ø=24 mm) glass microfiber filter
The ignition source was transferred under the sample and held in this its position for a preset time (1, 5 or 10 seconds) to ignite the sample. Ignition and burning of the sample were detected using a UV light detector.
Evaluation is carried out by plotting the burning time/weight of electrolyte [s g⁻¹] over ignition time [s] and extrapolation by linear regression line to ignition time=0 s Self-extinguishing time (s·g⁻¹) is the time that is needed until the sample stops burning once inflamed.

| | Concentration (% wt) in standard electrolyte 1M LiPF₆ in | | | | | |
|---|---|---|---|---|---|---|
| | (30% Ethylene carbonate & 70% ethyl methyl carbonate) | | | | | |
| | 0 | 2 | 5 | 10 | 30 | 100 |
| Component | Self-extinguishing time (s · g⁻¹) | | | | | |
| CF₃CH═CHOCH₂CF₃ | 39 ± 2 | 36 ± 5 | 38 ± 3 | 40 ± 3 | 37 ± 3 | 14 ± 2 |
| (MEXI-1) | | | | | | |
| CF₃CH═CHOCH₂CF₂CF₂H | 39 ± 2 | 47 ± 3 | 45 ± 2 | 43 ± 2 | 43 ± 2 | 25 ± 2 |
| (MEXI-2) | | | | | | |

These measurements demonstrate that the compounds MEXI-1 and MEXI-2 have flame retarding properties.

### Electrochemical Testing

### Drying

Before testing MEXI-1 and MEXI-2 were dried by treatment with a pre-activated type 4A molecular sieve. Water levels in the pre- and post-treated samples were determined by the Karl Fischer method:

| | Water level pre-treatment | Water level post-treatment |
|---|---|---|
| Compound | (ppm w/v) | (ppm w/v) |
| MEXI-1 | 76 | <10 |
| MEXI-2 | 103 | <10 |

### Electrolyte Formulation

Electrolyte preparation and storage was carried out in an argon filled glove box (H₂O and O₂<0.1 ppm). The base electrolyte was 1M LiPF₆ in ethylene carbonate:ethyl methyl carbonate (3:7 wt. %) with MEXI-1 or MEXI-2 additive at concentrations of 2, 5, 10 and 30 wt. %.

### Cell Chemistry and Construction

The performance of each electrolyte formulation was tested in multi-layer pouch cells over 50 cycles (2 cells per electrolyte):

Chemistry 1: Lithium-Nickel-Cobalt-Manganese-Oxide (NCM622) positive electrode and artificial graphite (specific capacity: 350 mAh g⁻¹) negative electrode. The area capacity of NMC622 and graphite amounted to 3.5 mAh cm⁻² and 4.0 mAh cm⁻², respectively. The N/P ratio amounted to 115%.

Chemistry 2: Lithium-Nickel-Cobalt-Manganese-Oxide (NCM622) positive electrode and SiOₓ/graphite (specific capacity: 550 mAh g⁻¹) negative electrode. The area capacity of NMC622 and SiOₓ/graphite amount to 3.5 mAh/cm⁻² and 4.0 mAh cm⁻², respectively. The N/P ratio amounted to 115%.

The test pouch cells had the following characteristics:
Nominal capacity 240 mAh+/−2%
Standard deviations:

Capacity: ±0.6 mAh

Coulombic Efficiency (CE) 1st cycle: ±0.13%

Coulombic Efficiency (CE) subsequent cycles: ±0.1%

Positive electrode: NMC-622
Active material content: 96.4%
Mass loading: 16.7 mg cm⁻²

Negative electrode: Artificial Graphite
Active material content: 94.8%
Mass loading: 10 mg cm⁻²
Separator: PE (16 μm)+4 μm Al₂O₃
Balanced at cut-off voltage of 4.2 V

Negative electrode: Artificial graphite+SiO
Active material content: 94.6%
Mass loading: 6.28 mg cm⁻²
Separator: PE (16 μm)+4 μm Al₂O₃
Balanced at cut-off voltage of 4.2 V

After assembly the following formation protocol was used:
1. Step charge to 1.5 V followed by 5 h rest step (wetting step @ 40° C.)
2. CCCV (C/10, 3.7 V (Iₗᵢₘᵢₜ: 1 h)) (preformation step)
3. Rest step (6 h)
4. CCCV (C/10, 4.2 V (Iₗᵢₘᵢₜ: 0.05 C)) rest step (20 min)
5. CC discharge (C/10, 3.8 V), (degassing of the cell)
6. CC discharge (C/10, 2.8 V)

Following this formation step, the cells were tested as follows:
Rest step (1.5 V, 5 h), CCCV (C/10, 3.7 V (1 h))
Rest step (6 h), CCCV (C/10, 4.2 V (Iₗᵢₘᵢₜ: 0.05 C))
Rest step (20 min), CC discharge (C/10, 3.8 V)
Degassing step
Discharge (C/10, 2.8 V), rest step (5 h)
CCCV (C/3, 4.2 V (Iₗᵢₘᵢₜ: 0.05 C)), rest step (20 min)
CC discharge (C/3, 2.8 V)
50 cycles or until 50% SOH is reached at 40° C.:

CCCV (C/3, 4.2 V (Iₗᵢₘᵢₜ: 0.02 C)), rest step (20 min)

CC discharge (C/3, 3.0 V), rest step (20 min)

### Test Results

The test results for both additives in each cell chemistry are summarised in Tables 5-8 and FIGS. 24-27. From this data it can be seen that both additives in both cell chemistries had a positive influence on cell performance improving both Coulombic efficiency and cycling stability. These results combined with the safety related studies demonstrate that the compounds of this invention simultaneously improved both the safety and performance of energy storage devices containing them.

TABLE 5
Electrochemical performance of MEXI-1 - Cell Chemistry 1

| | | | Base electrolyte + | | Base electrolyte + | | Base electrolyte + | | Base electrolyte + | |
|---|---|---|---|---|---|---|---|---|---|---|
| | Base electrolyte | | 2 wt. % MEXI-1 | | 5 wt. % MEXI-1 | | 10 wt. % MEXI-1 | | 30 wt. % MEXI-1 | |
| | Discharge | Coulombic | Discharge | Coulombic | Discharge | Coulombic | Discharge | Coulombic | Discharge | Coulombic |
| Cycle | capacity | efficiency | capacity | efficiency | capacity | efficiency | capacity | efficiency | capacity | efficiency |
| No. | (mAh) | (%) | (mAh) | (%) | (mAh) | (%) | (mAh) | (%) | (mAh) | (%) |
| 1^{st} | 232.2 | 90.6 | 239.6 | 90.6 | 241.1 | 90.9 | 237.8 | 90.1 | 238.7 | 90.9 |
| (0.1 C) | | | | | | | | | | |
| 3^{rd} | 224.4 | 99.6 | 233.3 | 99.5 | 233.1 | 99.5 | 231.4 | 99.5 | 231.9 | 99.6 |
| (0.3 C) | | | | | | | | | | |
| 50^{th} | 218.1 | 99.8 | 227.0 | 99.8 | 226.8 | 99.8 | 224.8 | 99.8 | 226.8 | 99.9 |
| (0.3 C) | | | | | | | | | | |

TABLE 6
Electrochemical performance of MEXI-1 - Cell Chemistry 2

| | | | Base electrolyte + | | Base electrolyte + | | Base electrolyte + | | Base electrolyte + | |
|---|---|---|---|---|---|---|---|---|---|---|
| | Base electrolyte | | 2 wt. % MEXI-1 | | 5 wt. % MEXI-1 | | 10 wt. % MEXI-1 | | 30 wt. % MEXI-1 | |
| | Discharge | Coulombic | Discharge | Coulombic | Discharge | Coulombic | Discharge | Coulombic | Discharge | Coulombic |
| Cycle | capacity | efficiency | capacity | efficiency | capacity | efficiency | capacity | efficiency | capacity | efficiency |
| No. | (mAh) | (%) | (mAh) | (%) | (mAh) | (%) | (mAh) | (%) | (mAh) | (%) |
| 1^{st} | 199.6 | 74.5 | 199.0 | 74.6 | 199.2 | 74.7 | 200.1 | 74.6 | 201.8 | 75.3 |
| (0.1 C) | | | | | | | | | | |
| 3^{rd} | 176.3 | 97.0 | 176.3 | 97.1 | 175.4 | 96.9 | 177.3 | 97.2 | 181.4 | 97.6 |
| (0.3 C) | | | | | | | | | | |
| 50^{th} | 125.7 | 99.6 | 125.9 | 99.6 | 125.8 | 99.6 | 127.8 | 99.6 | 134.3 | 99.6 |
| (0.3 C) | | | | | | | | | | |

TABLE 7
Electrochemical performance of MEXI-2 - Cell Chemistry 1

| | | | Base electrolyte + | | Base electrolyte + | | Base electrolyte + | | Base electrolyte + | |
|---|---|---|---|---|---|---|---|---|---|---|
| | Base electrolyte | | 2 wt. % MEXI-1 | | 5 wt. % MEXI-1 | | 10 wt. % MEXI-1 | | 30 wt. % MEXI-1 | |
| | Discharge | Coulombic | Discharge | Coulombic | Discharge | Coulombic | Discharge | Coulombic | Discharge | Coulombic |
| Cycle | capacity | efficiency | capacity | efficiency | capacity | efficiency | capacity | efficiency | capacity | efficiency |
| No. | (mAh) | (%) | (mAh) | (%) | (mAh) | (%) | (mAh) | (%) | (mAh) | (%) |
| 1^{st} | 232.2 | 90.6 | 239.5 | 90.8 | 239.8 | 90.6 | 239.1 | 90.5 | 240.5 | 91.1 |
| (0.1 C) | | | | | | | | | | |
| 3^{rd} | 224.4 | 99.6 | 231.5 | 99.6 | 232.0 | 99.5 | 232.3 | 99.6 | 233.0 | 99.6 |
| (0.3 C) | | | | | | | | | | |
| 50^{th} | 218.1 | 99.8 | 225.6 | 99.8 | 225.6 | 99.8 | 226.2 | 99.8 | 227.9 | 99.9 |
| (0.3 C) | | | | | | | | | | |

TABLE 8
Electrochemical performance of MEXI-2 - Cell Chemistry 2

| | | | Base electrolyte + | | Base electrolyte + | | Base electrolyte + | | Base electrolyte + | |
|---|---|---|---|---|---|---|---|---|---|---|
| | Base electrolyte | | 2 wt. % MEXI-1 | | 5 wt. % MEXI-1 | | 10 wt. % MEXI-1 | | 30 wt. % MEXI-1 | |
| | Discharge | Coulombic | Discharge | Coulombic | Discharge | Coulombic | Discharge | Coulombic | Discharge | Coulombic |
| Cycle | capacity | efficiency | capacity | efficiency | capacity | efficiency | capacity | efficiency | capacity | efficiency |
| No. | (mAh) | (%) | (mAh) | (%) | (mAh) | (%) | (mAh) | (%) | (mAh) | (%) |
| 1^{st} | 199.6 | 74.5 | 201.6 | 74.8 | 201.1 | 74.8 | 198.0 | 74.4 | 200.8 | 75.3 |
| (0.1 C) | | | | | | | | | | |
| 3^{rd} | 176.3 | 97.0 | 177.9 | 97.0 | 178.3 | 97.0 | 176.7 | 97.3 | 179.8 | 97.5 |
| (0.3 C) | | | | | | | | | | |
| 50^{th} | 125.7 | 99.6 | 126.8 | 99.7 | 127.6 | 99.6 | 126.9 | 99.6 | 132.6 | 99.7 |
| (0.3 C) | | | | | | | | | | |

### FIGURES

FIG. 1 shows three CV traces which serve to demonstrate the electrochemical compatibility of trifluoropropenyl ethers such as product E6.

FIGS. 2-11 illustrate the results of various spectroscopic analytical techniques carried out on compositions comprising some of the reaction products from the Examples and some reference products.

FIG. 2 shows a ¹⁹F NMR spectrum of LiPF₆ in ethylene carbonate.

FIG. 3 shows a ¹⁹F NMR spectrum of LiPF₆ in propylene carbonate.

FIG. 4 shows a ¹⁹F NMR spectrum of LiPF₆ in ethylene carbonate/propylene carbonate/dimethyl carbonate.

FIG. 5 shows a ¹⁹F NMR spectrum of LiPF₆ in 30% E6/70% ethylene carbonate.

FIG. 6 shows a ¹⁹F NMR spectrum of LiPF₆ in 30% E6/70% propylene carbonate.

FIG. 7 shows a ¹⁹F NMR spectrum of LiPF₆ in 80% E6/20% ethylene carbonate.

FIG. 8 shows a ¹⁹F NMR spectrum of LiPF₆ in 80% E6/20% propylene carbonate.

FIG. 9 shows a ¹⁹F NMR spectrum of LiPF₆ in 30% E7/70% propylene carbonate.

FIG. 10 shows a ¹⁹F NMR spectrum of LiPF₆ in 50% E7/50% ethylene carbonate.

FIG. 11 shows a ¹⁹F NMR spectrum of LiPF₆ in 80% E7/20% ethylene carbonate.

FIGS. 12a to 12d show ¹⁹F NMR spectra of LiPF₆ and CF₃CH═CHOCH₂CF₃ in propylene carbonate.

FIGS. 13a to 13c show ¹⁹F NMR spectra of LiPF₆ and CF₃CH═CHOCH₂CF₃ in propylene carbonate (90%) and fluoroethylene carbonate (10%).

FIGS. 14a to 14c show ¹⁹F NMR spectra of LiPF₆ and CF₃CH═CHOCH₂CF₃ in ethylene carbonate (30%) and ethyl methyl carbonate (70%).

FIGS. 15a to 15c show ¹⁹F NMR spectra of LiFSI and CF₃CH═CHOCH₂CF₃ in propylene carbonate.

FIGS. 16a to 16c show ¹⁹F NMR spectra of LiFSI and CF₃CH═CHOCH₂CF₃ in propylene carbonate (90%) and fluoroethylene carbonate (10%).

FIGS. 17a to 17b show ¹⁹F NMR spectra of LiFSI and CF₃CH═CHOCH₂CF₃ in ethylene carbonate (30%) and ethyl methyl carbonate (70%).

FIGS. 18a to 18c show ¹⁹F NMR spectra of LiPF₆ and CF₃CH═CHOCH₂CF₂CHF₂ in propylene carbonate.

FIGS. 19a to 19c show ¹⁹F NMR spectra of LiPF₆ and CF₃CH═CHOCH₂CF₂CHF₂ in propylene carbonate (90%) and fluoroethylene carbonate (10%).

FIGS. 20a to 20c show ¹⁹F NMR spectra of LiPF₆ and CF₃CH═CHOCH₂CF₂CHF₂ in ethylene carbonate (30%) and ethyl methyl carbonate (70%).

FIGS. 21a to 21c show ¹⁹F NMR spectra of LiFSI and CF₃CH═CHOCH₂CF₂CHF₂ in propylene carbonate.

FIGS. 22a to 22c show ¹⁹F NMR spectra of LiFSI and CF₃CH═CHOCH₂CF₂CHF₂ in propylene carbonate (90%) and fluoroethylene carbonate (10%).

FIGS. 23a to 23c show ¹⁹F NMR spectra of LiFSI and CF₃CH═CHOCH₂CF₂CHF₂ in ethylene carbonate (30%) and ethyl methyl carbonate (70%).

FIG. 24 shows the electrochemical performance of MEXI-1—cell chemistry 1

FIG. 25 shows the electrochemical performance of MEXI-1—cell chemistry 2

FIG. 26 shows the electrochemical performance of MEXI-2—cell chemistry 1

FIG. 27 shows the electrochemical performance of MEXI-2—cell chemistry 2 (% in composition by weight)

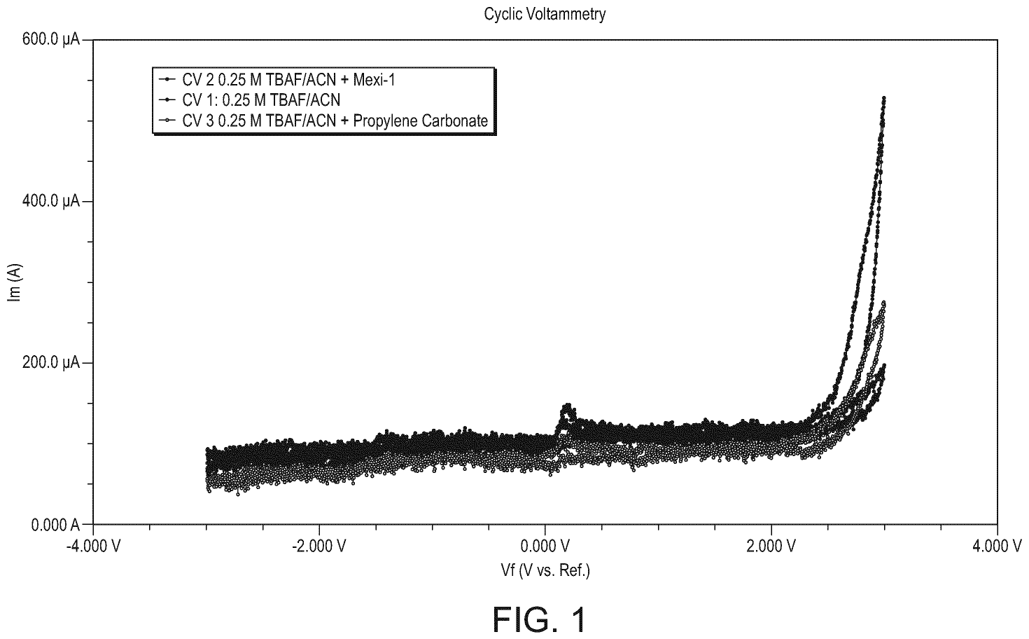

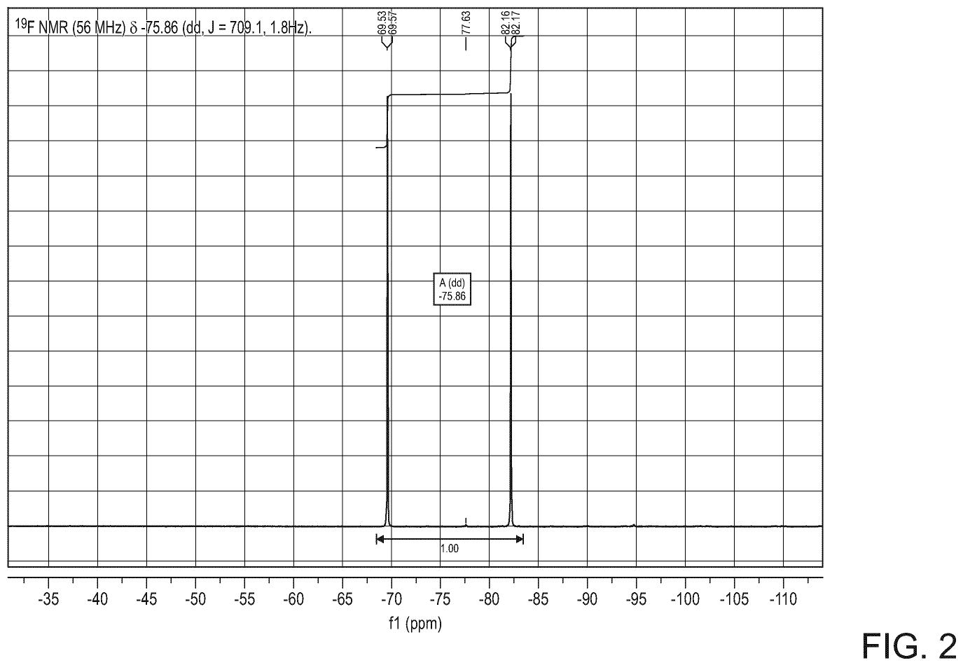

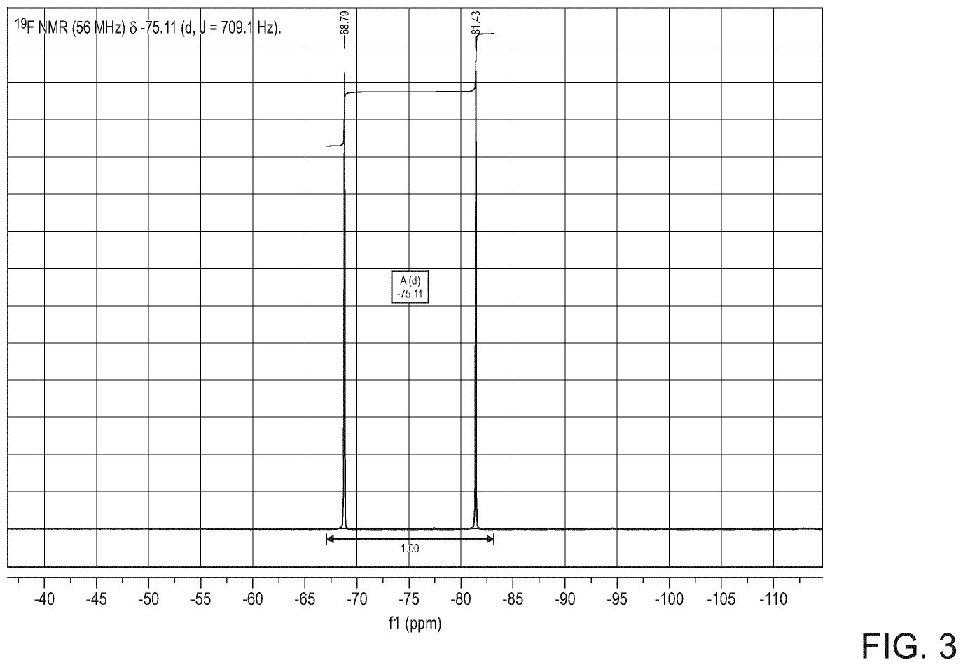

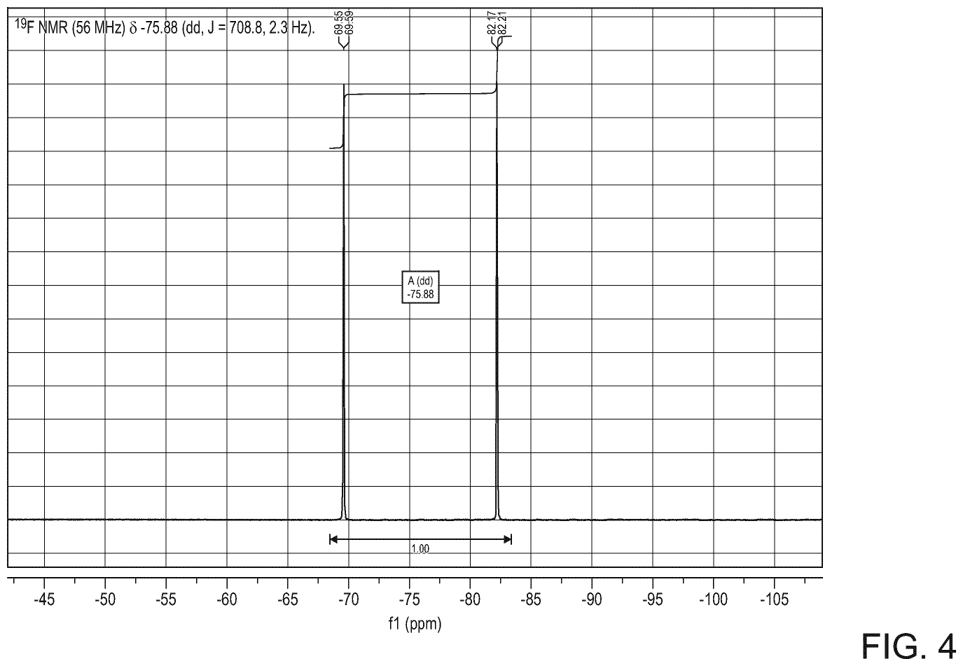

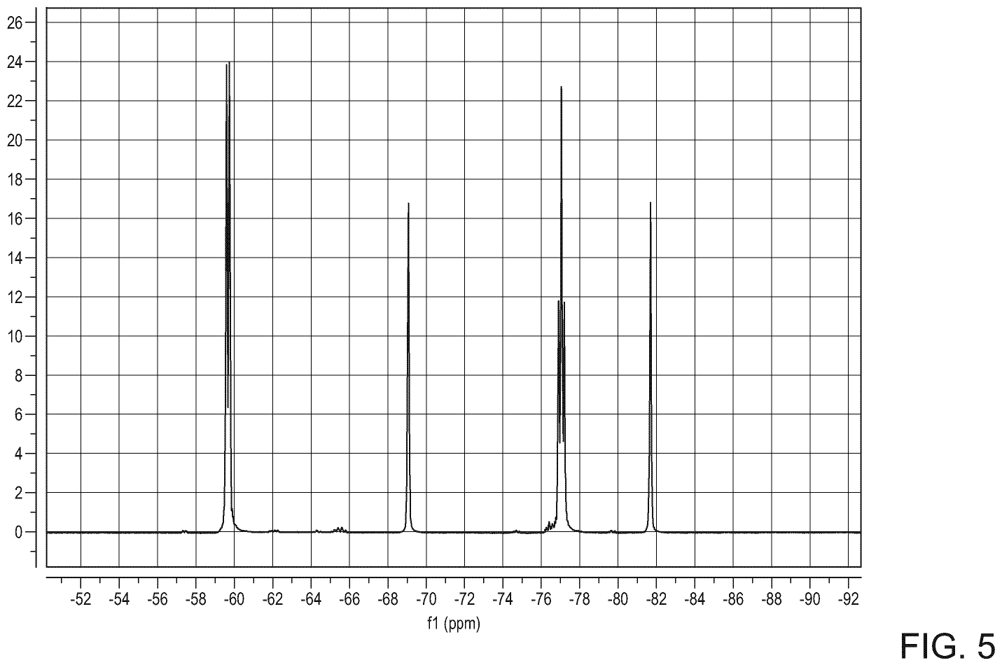

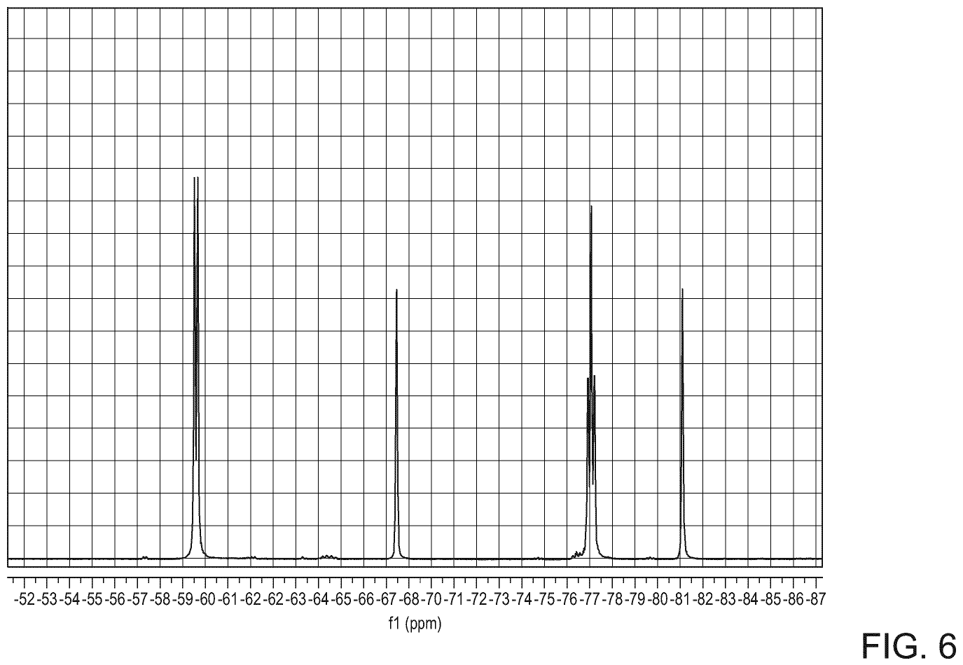

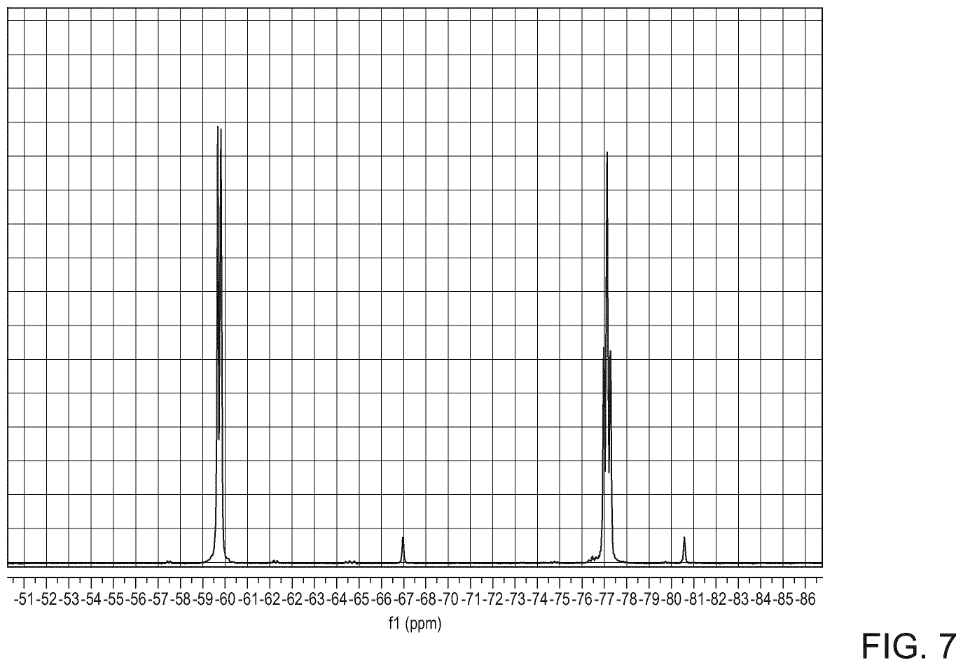

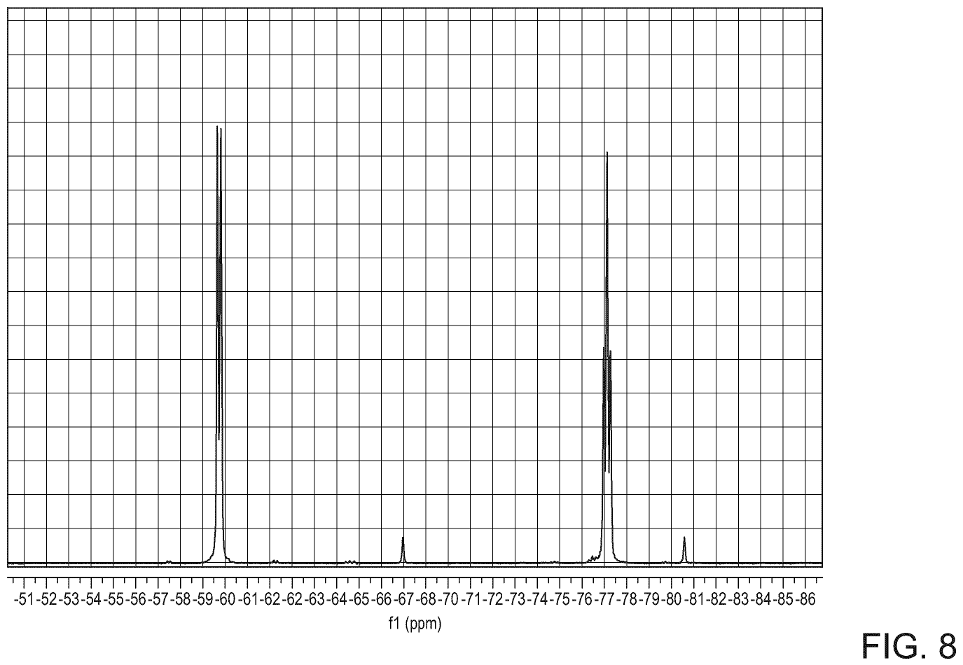

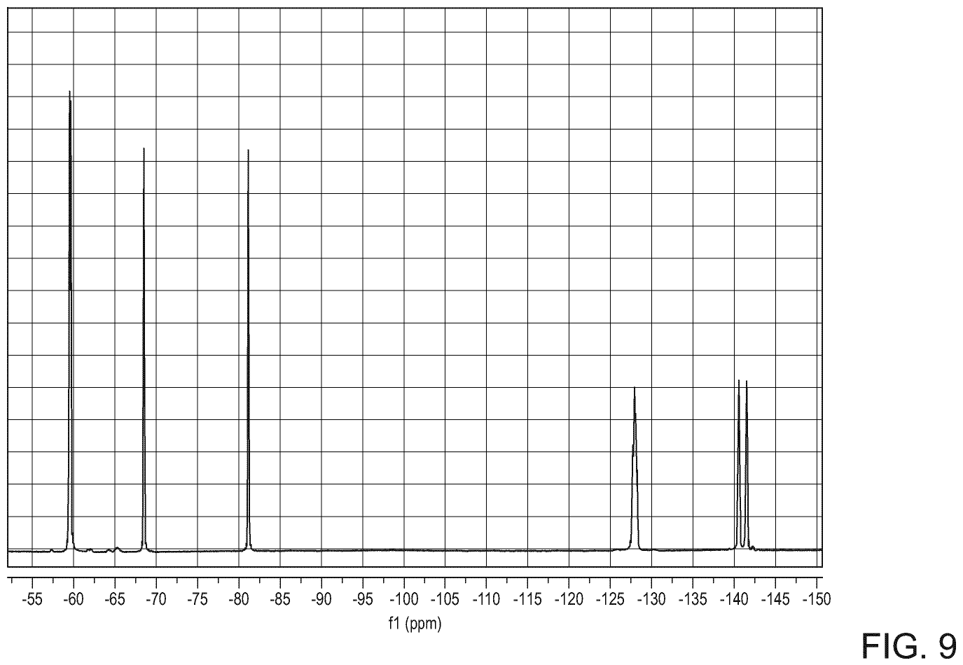

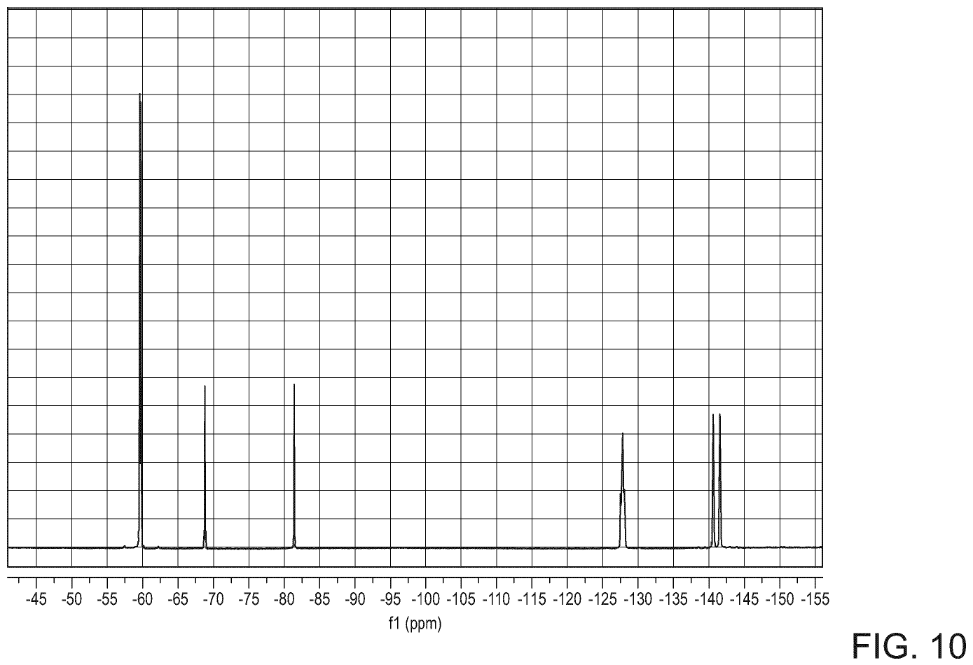

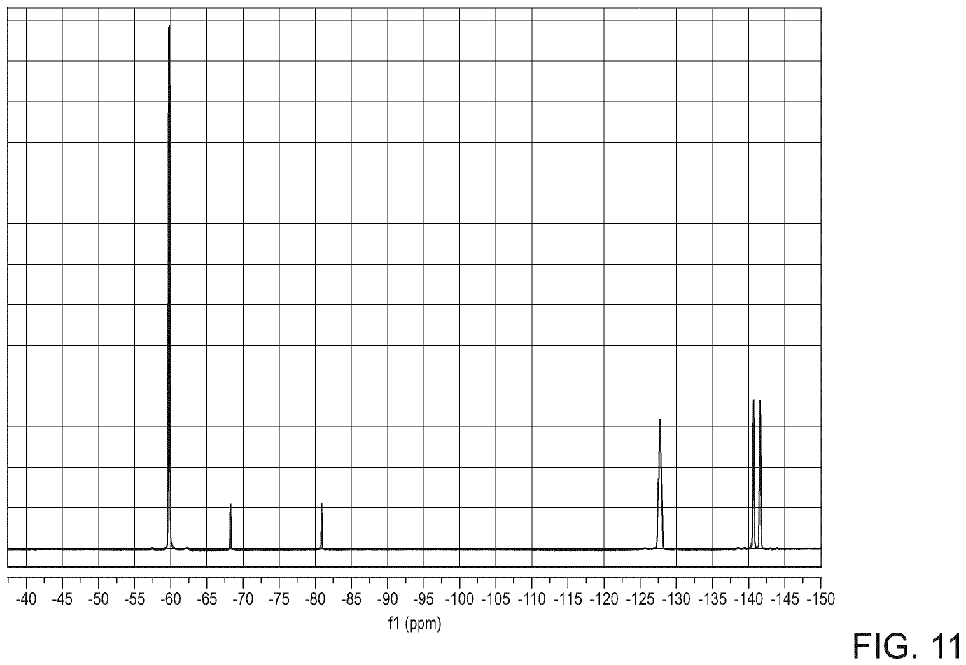

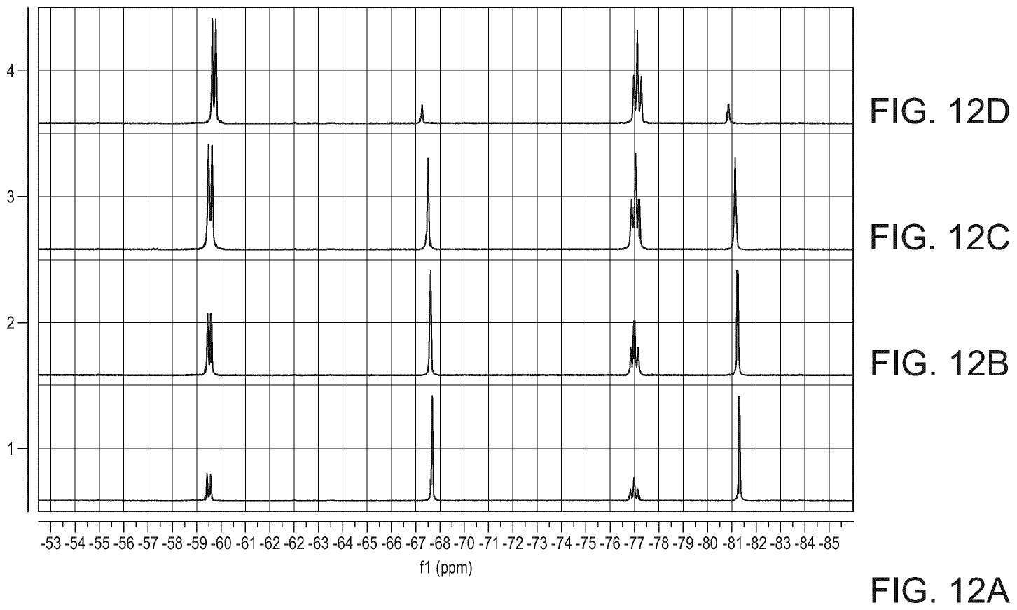

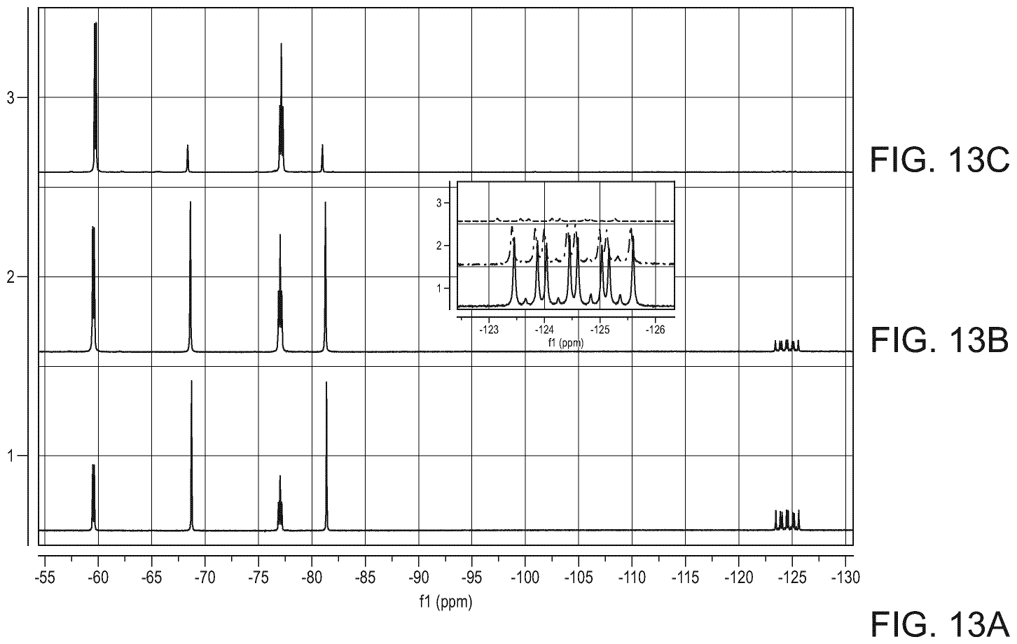

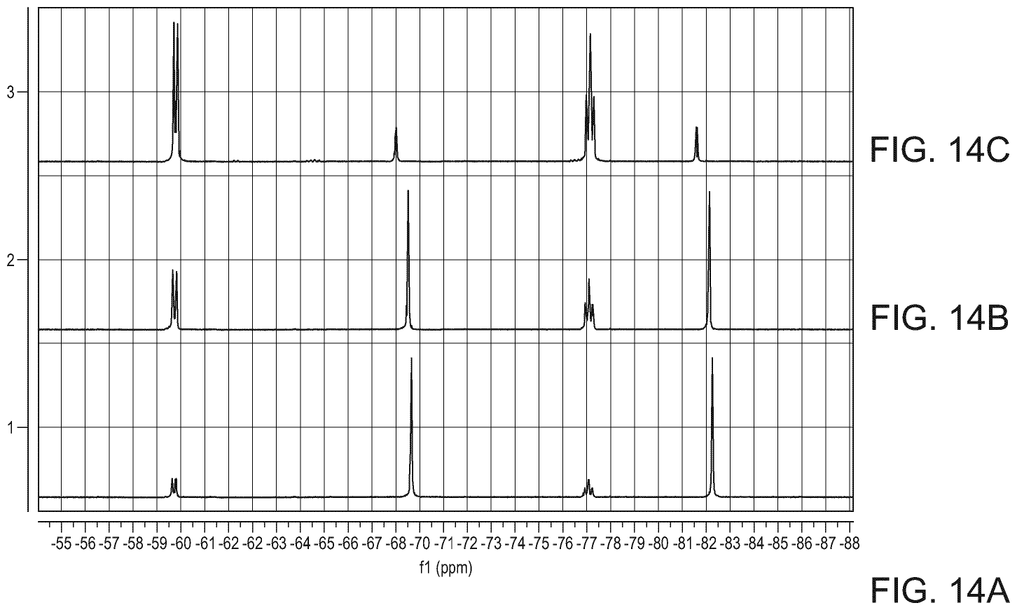

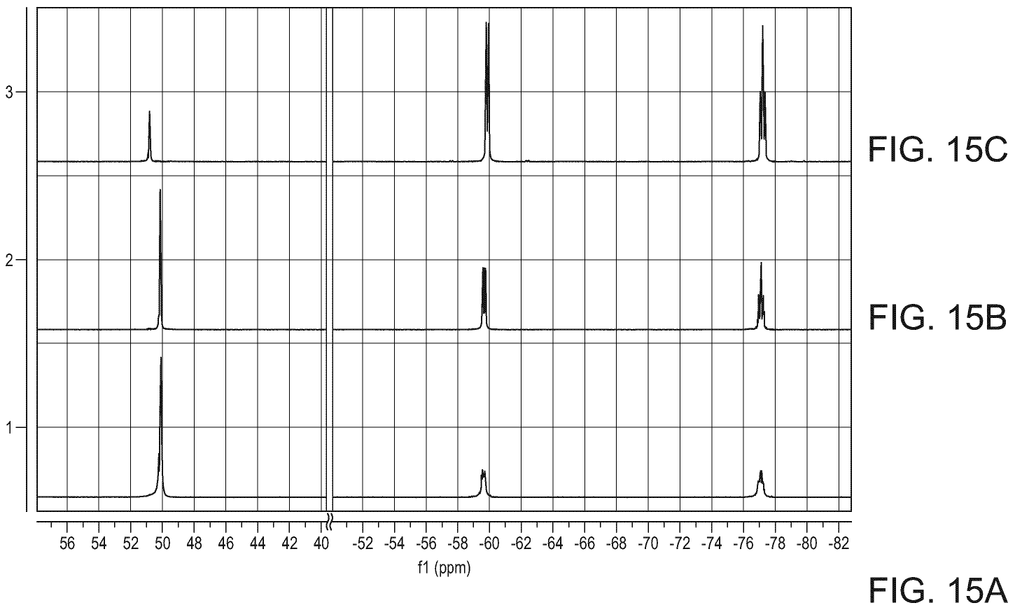

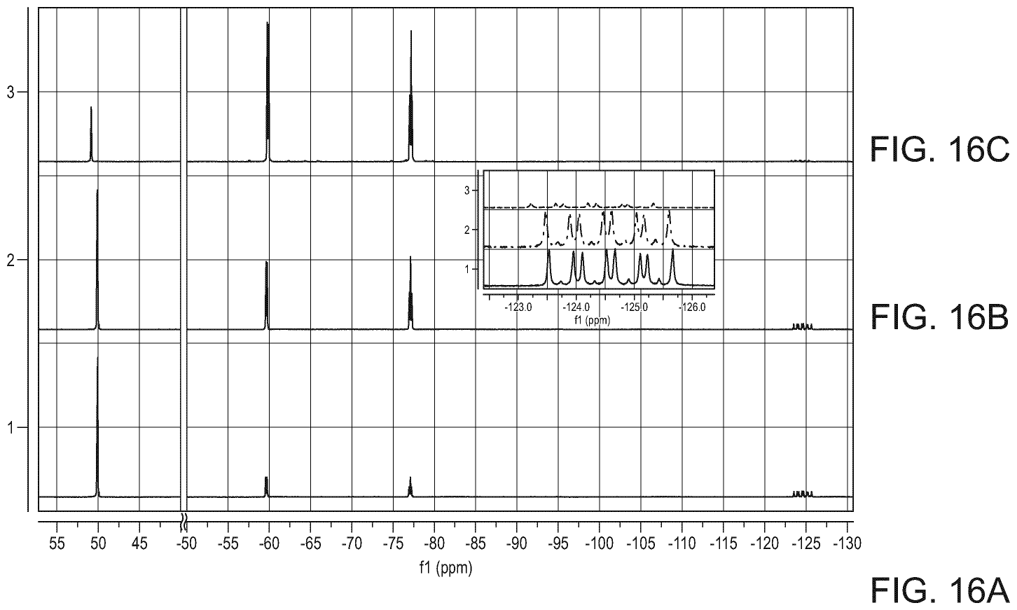

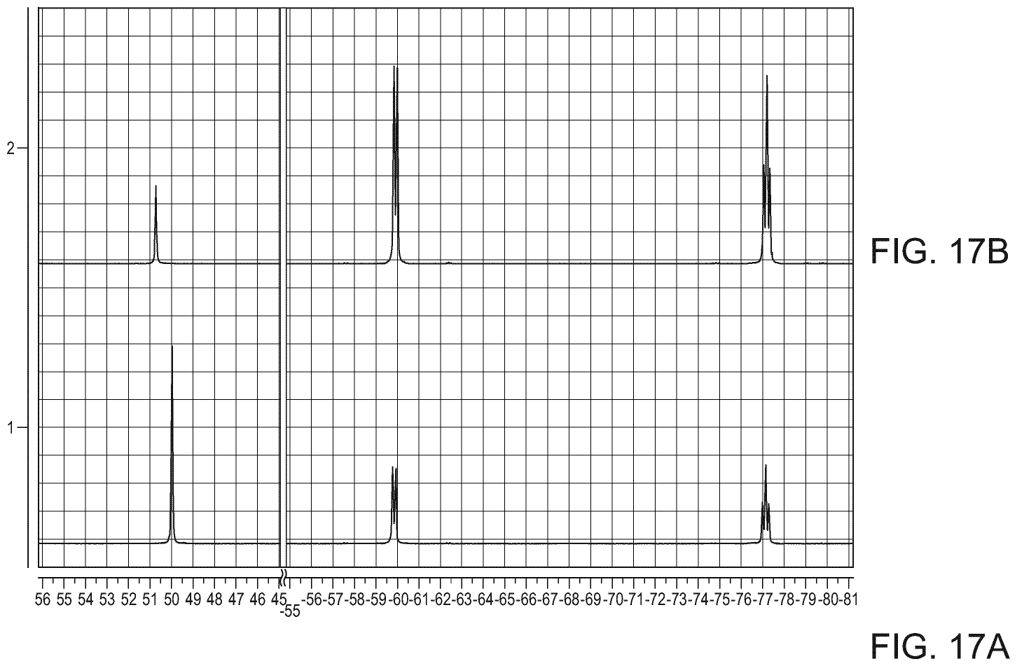

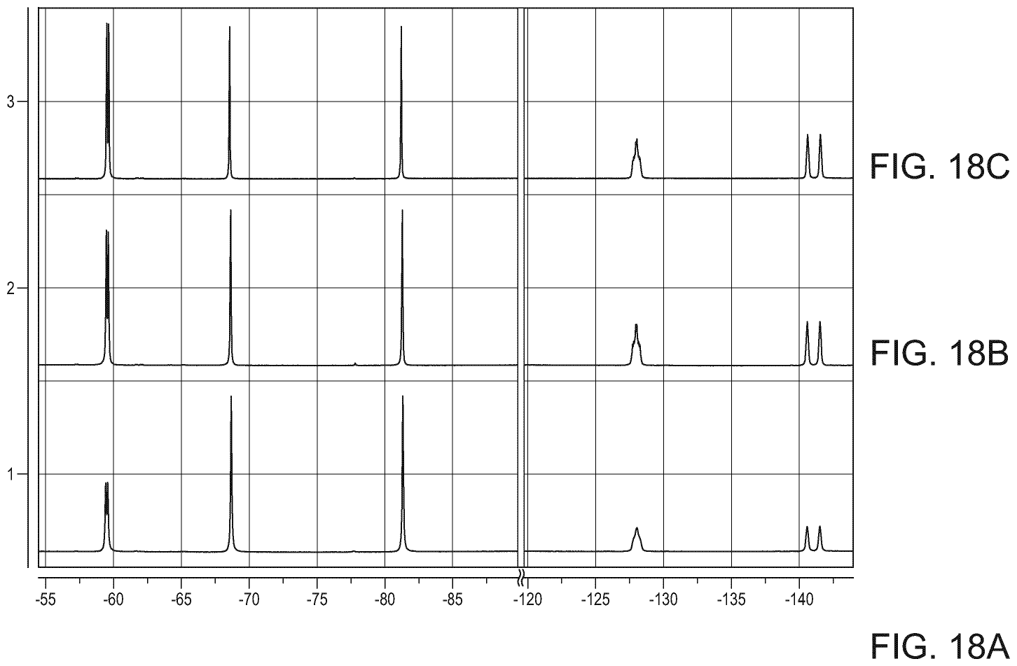

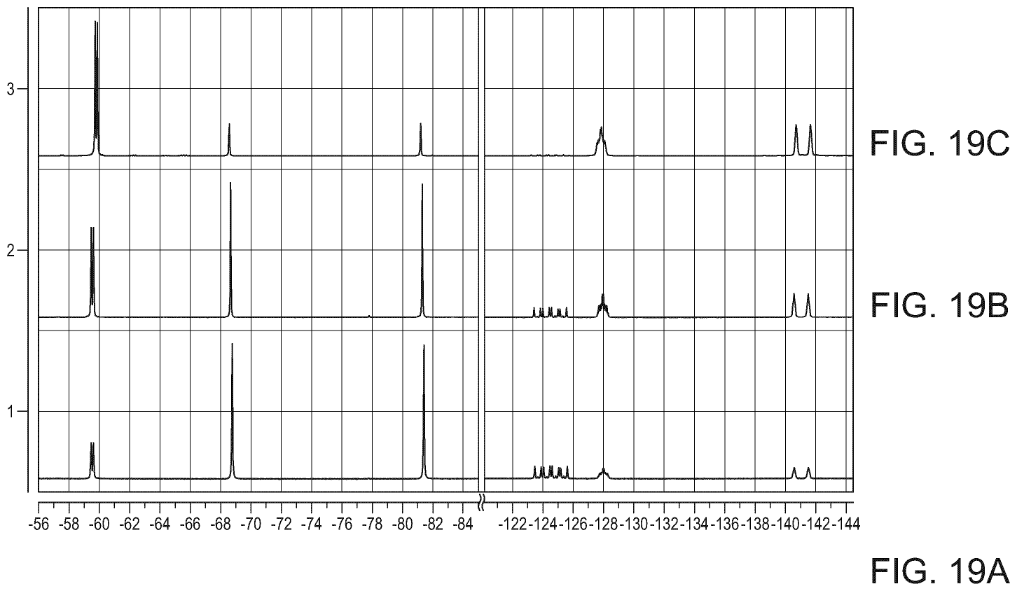

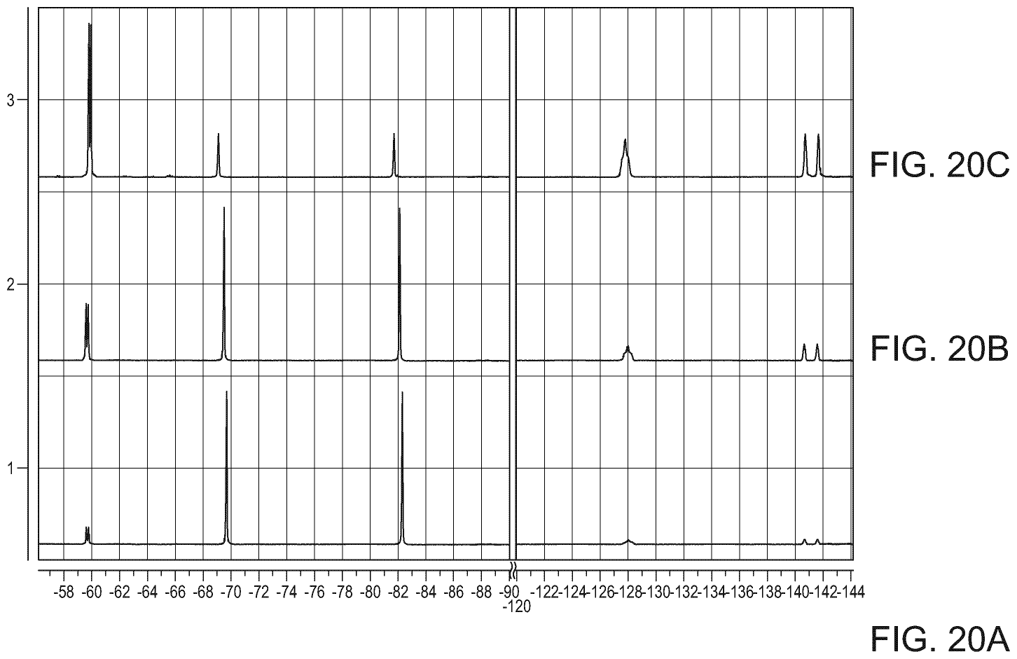

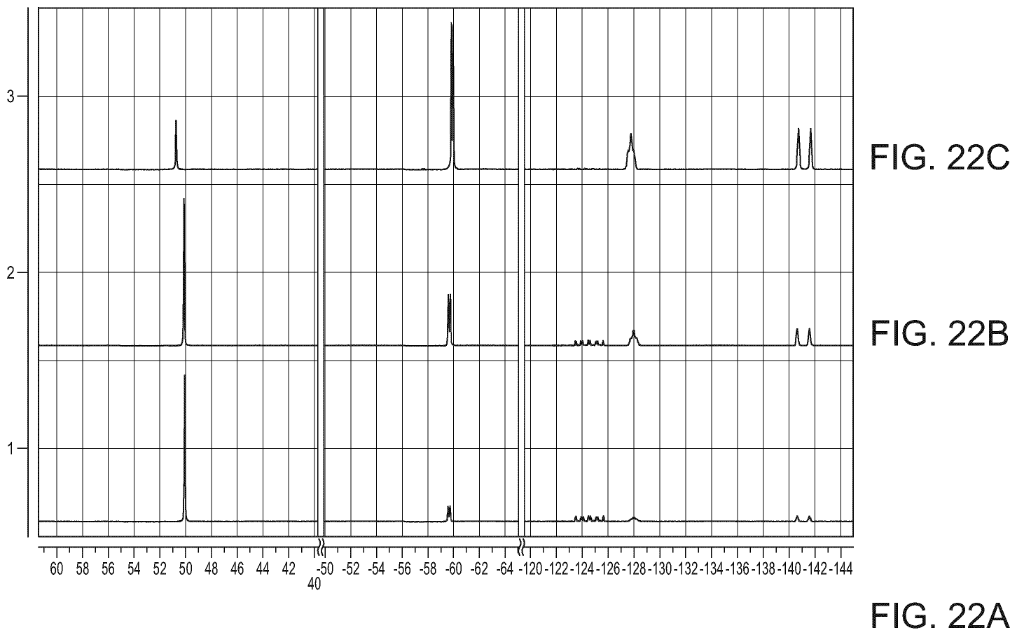

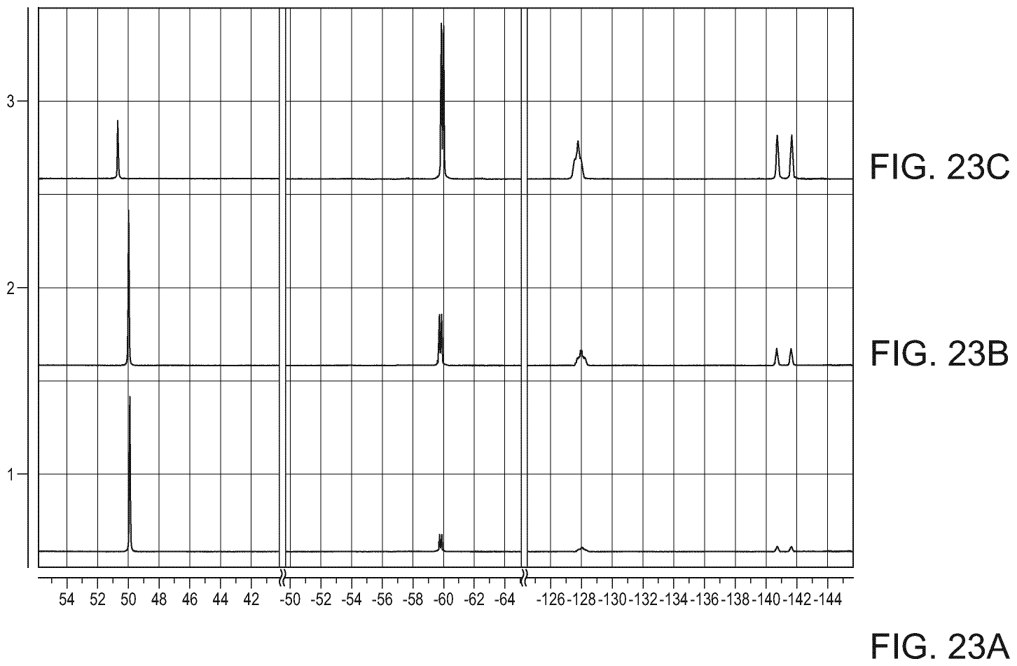

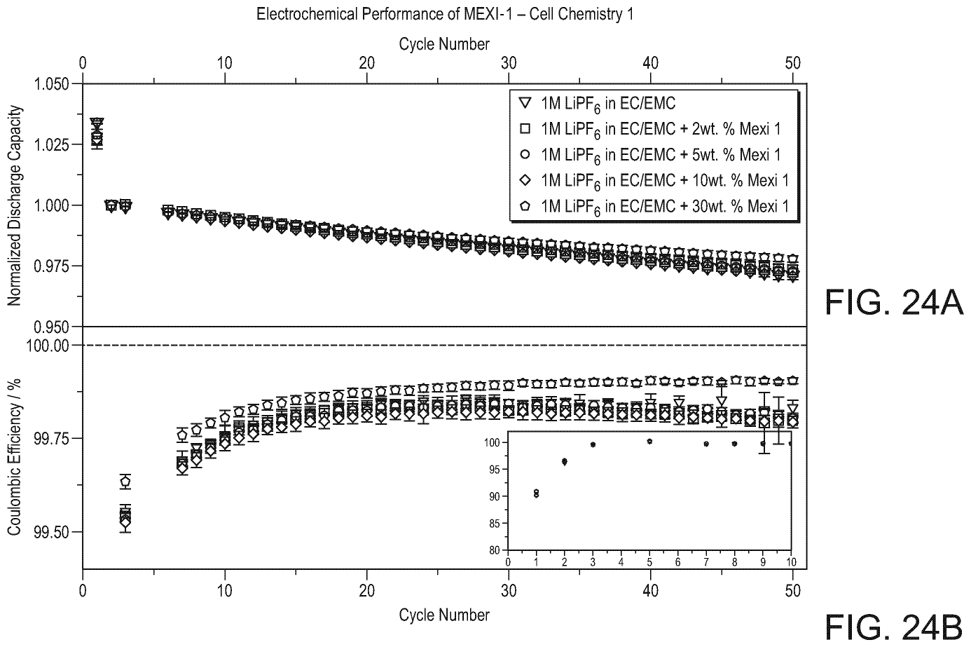

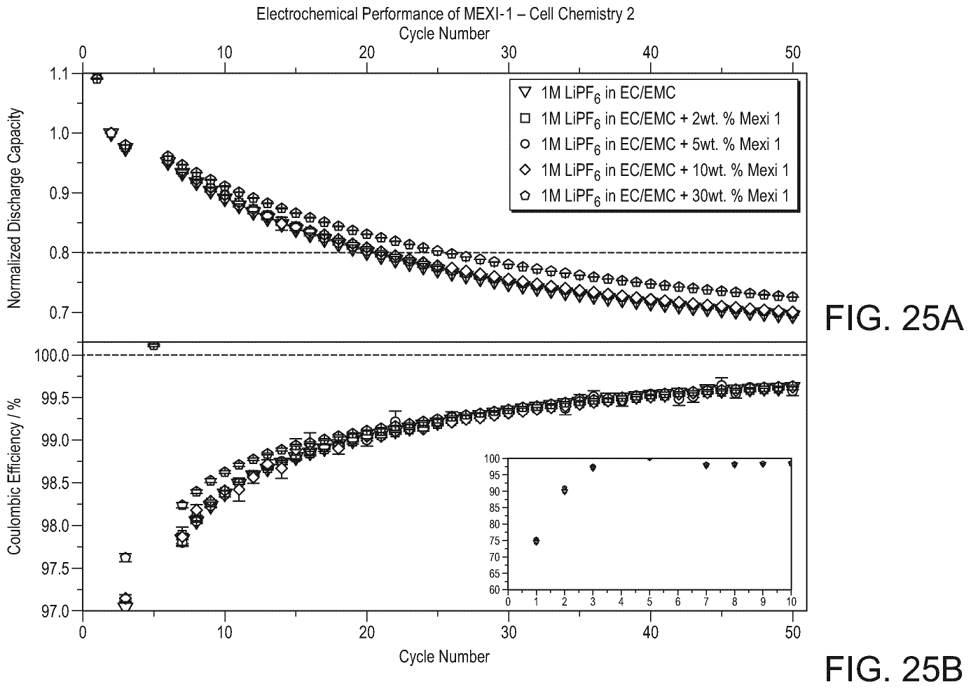

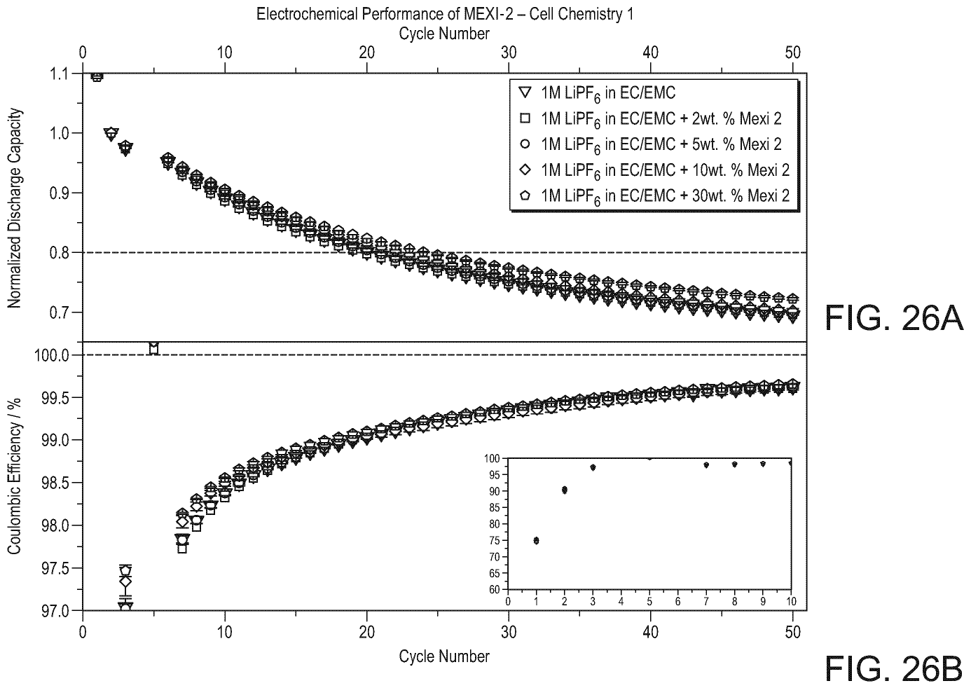

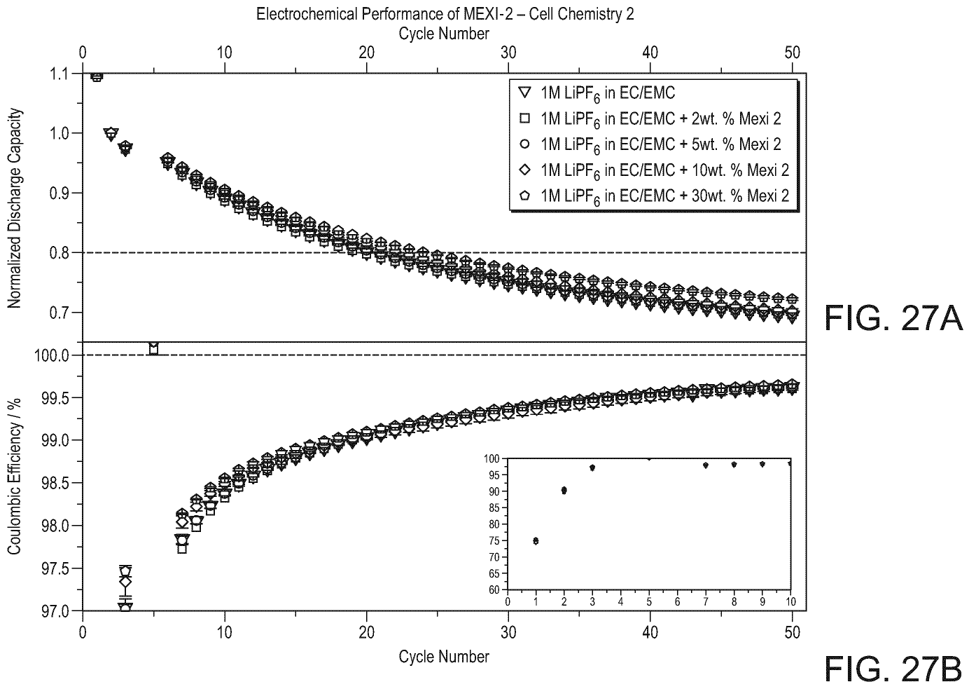

1. A nonaqueous battery electrolyte formulation, comprising a compound of Formula 1: 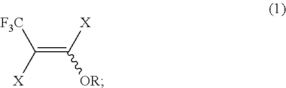
wherein R is a fluorinated alkyl group; and
X is selected from the group consisting of F, Cl, H, CF₃, and C₁ to C₆ alkyl which is at least partially fluorinated; and
OR is cis- or trans- to any vicinal group.

2. A battery, comprising the battery electrolyte formulation according to claim 1.

3. The formulation according to claim 1, further comprising a metal electrolyte salt, present in an amount of 0.1 to 20 wt % relative to the total mass of the battery electrolyte formulation, and wherein the metal electrolyte salt is a salt of lithium, sodium, magnesium, calcium, lead, zinc, or nickel.

4. (canceled)

5. The formulation according to claim 3, wherein the metal salt is a salt of lithium selected from the group consisting of lithium hexafluorophosphate (LiPF₆), lithium hexafluoroarsenate monohydrate (LiAsF₆), lithium perchlorate (LiClO₄), lithium tetrafluoroborate (LiBF₄), lithium triflate (LiSO₃CF₃), lithium bis(fluorosulfonyl)imide (Li(FSO₂)₂N), and lithium bis(trifluoromethanesulfonyl)imide (Li(CF₃SO₂)₂N).

6. The formulation according to claim 1, further comprising an additional solvent in an amount of from 0.1 wt % to 99.9 wt % of the liquid component of the formulation, wherein the additional solvent is selected from the group consisting of dimethyl carbonate (DMC), ethylmethyl carbonate (EMC), fluoroethylene carbonate (FEC), propylene carbonate (PC), and ethylene carbonate (EC).

7-8. (canceled)

9. A formulation comprising a metal ion and a compound of Formula 2, optionally in combination with a solvent: 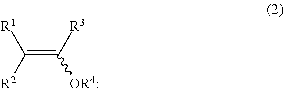
wherein R¹, R², and R³ are each independently selected from the group consisting of F, Cl, H, CF₃, and C₁ to C₆ alkyl which is at least partially fluorinated; and
R⁴ is selected from the group consisting of C₁ to C₁₂ alkyl which may be at least partially fluorinated; and
OR⁴ is cis- or trans- to R¹ or R².

10. A battery comprising the formulation according to claim 9.

11. The formulation according to claim 9, further comprising a metal electrolyte salt, present in an amount of 0.1 to 20 wt % relative to the total mass of the formulation, wherein the metal electrolyte salt is a salt of lithium, sodium, magnesium, calcium, lead, zinc, or nickel.

12. (canceled)

13. The formulation according to claim 11, wherein the metal electrolyte salt is a salt of lithium selected from the group consisting of lithium hexafluorophosphate (LiPF₆), lithium hexafluoroarsenate monohydrate (LiAsF₆), lithium perchlorate (LiClO₄), lithium tetrafluoroborate (LiBF₄), lithium triflate (LiSO₃CF₃), lithium bis(fluorosulfonyl)imide (Li(FSO₂)₂N), and lithium bis(trifluoromethanesulfonyl)imide (Li(CF₃SO₂)₂N).

14. The formulation according to claim 9, further comprising an additional solvent in an amount of from 0.1 wt % to 99.9 wt % of the liquid component of the formulation, wherein the additional solvent is selected from the group consisting of dimethyl carbonate (DMC), ethylmethyl carbonate (EMC), fluoroethylene carbonate (FEC), propylene carbonate (PC), and ethylene carbonate (EC).

15. (canceled)

16. A method of reducing the flammability of a battery and/or a battery electrolyte comprising adding to the battery and/or the battery electrolyte the battery electrolyte formulation according to claim 1.

17. A method of powering an article comprising a battery, the method comprising adding to the battery the battery electrolyte formulation according to claim 1.

18. A method of retrofitting a battery electrolyte comprising (a) at least partially replacing the battery electrolyte with the battery electrolyte formulation according to claim 1 and/or (b) supplementing the battery electrolyte with the battery electrolyte formulation.

19. A method of preparing a compound of Formula 1 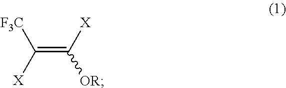
wherein R is a fluorinated alkyl group;
X is selected from the group consisting of F, Cl, H, CF₃, and C₁ and C₆ alkyl which is at least partially fluorinated; and
—OR is cis- or trans- to any vicinal group;
the method comprising reacting a compound of Formula 2a and/or Formula 2b: 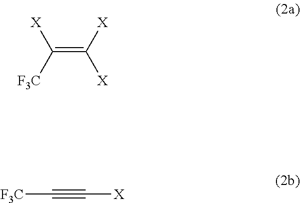
with an alcohol of formula ROH under basic conditions at elevated temperature and pressure so as to produce the compound of Formula 1.

20. A method of preparing the battery electrolyte formulation according to claim 5 comprising mixing a compound of Formula 1 with ethylene, propylene, or fluoroethylene carbonate and with the salt of lithium so as to produce the formulation.

21. The method according to claim 17, wherein a capacity of the battery and/or charge transfer within the battery is improved relative to a battery without the formulation.

22. The method according to claim 16, wherein the formulation comprises a metal electrolyte salt, present in an amount of 0.1 to 20 wt % relative to the total mass of the nonaqueous electrolyte formulation, wherein the metal electrolyte salt is a salt of lithium, sodium, magnesium, calcium, lead, zinc, or nickel.

23. (canceled)

24. A The method according to claim 22, wherein the metal electrolyte salt is a salt of lithium selected from the group consisting of lithium hexafluorophosphate (LiPF₆), lithium hexafluoroarsenate monohydrate (LiAsF₆), lithium perchlorate (LiClO₄), lithium tetrafluoroborate (LiBF₄), lithium triflate (LiSO₃CF₃), lithium bis(fluorosulfonyl)imide (Li(FSO₂)₂N), and lithium bis(trifluoromethanesulfonyl)imide (Li(CF₃SO₂)₂N).

25. A The method according to claim 16, wherein the formulation comprises an additional solvent in an amount of from 0.1 wt % to 99.9 wt % of a liquid component of the formulation, wherein the additional solvent is selected from the group consisting of dimethyl carbonate (DMC), ethylmethyl carbonate (EMC), fluoroethylene carbonate (FEC), propylene carbonate (PC), and ethylene carbonate (EC).

26. (canceled)

27. A method of powering an article comprising a battery, the method comprising adding to the battery the formulation according to claim 7.
